(19) United States
(12) Patent Application Publication
(10) Pub. No.: US 2014/0066835 A1
(43) Pub. Date: Mar. 6, 2014

(54) SYSTEMS AND METHODS FOR CORNEAL CROSS-LINKING WITH PULSED LIGHT

(71) Applicant: Avedro, Inc., Waltham, MA (US)

(72) Inventors: David Muller, Boston, MA (US); Marc D. Friedman, Needham, MA (US); John Kanellopoulos, New York, NY (US)

(73) Assignee: Avedro, Inc., Waltham, MA (US)

(21) Appl. No.: 14/072,640

(22) Filed: Nov. 5, 2013

Related U.S. Application Data
(63) Continuation-in-part of application No. 13/480,127, filed on May 24, 2012.
Application No. 13/480,127 is a continuation-in-part of application No. 13/841,617, filed on Mar. 15, 2013.
Application No. 13/841,617 is a continuation-in-part of application No. 13/665,495, filed on Oct. 31, 2012.
(60) Provisional application No. 61/489,554, filed on May 24, 2011.
(60) Provisional application No. 61/492,499, filed on Jun. 2, 2011.
(60) Provisional application No. 61/699,226, filed on Sep. 10, 2012.
(60) Provisional application No. 61/671,798, filed on Jul. 16, 2012.
(60) Provisional application No. 61/722,613, filed on Nov. 5, 2012.

(51) Int. Cl. A61N 5/06; A61F 9/008

(52) U.S. Cl. CPC A61N 5/062; A61F 9/00804; A61F 9/00836
USPC 604/20

(57) ABSTRACT

Systems and methods for treating an eye select locations for making incisions in areas of the cornea according to astigmatic keratotomy or radial keratotomy, make incisions in the selected areas of the cornea, apply a cross-linking agent to the selected areas of the cornea, and deliver photoactivating light from a light source to the selected areas of the cornea to initiate cross-linking activity in the selected areas of the cornea.

### CROSS REFERENCE TO RELATED APPLICATIONS

This application is a continuation-in-part of U.S. patent application Ser. No. 13/480,127, filed May 24, 2012, which claims priority to U.S. Provisional Application No. 61/489,554, filed May 24, 2011, and U.S. Provisional Application No. 61/492,499, filed Jun. 2, 2011. This application is also a continuation-in-part of U.S. patent application Ser. No. 13/841,617, filed Mar. 15, 2013, which claims priority to U.S. Provisional Application No. 61/699,226, filed Sep. 10, 2012 and U.S. Provisional Application No. 61/671,798, filed Jul. 16, 2012, and is a continuation-in-part of U.S. patent application Ser. No. 13/665,495, filed Oct. 31, 2012. This application also claims priority to U.S. Provisional Application No. 61/722,613, filed Nov. 5, 2012. The contents of these applications are all incorporated entirely herein by reference.

### BACKGROUND OF THE INVENTION

1. Field of the Invention

The present invention relates generally to systems and methods for conducting an eye treatment, and more particularly to systems and methods for achieving corrective changes in corneal tissue and improving the stability of the changes to the corneal tissue.

2. Description of Related Art

A variety of eye disorders, such as myopia, keratoconus, and hyperopia, involve abnormal shaping of the cornea. Laser-assisted in-situ keratomileusis (LASIK) is one of a number of corrective procedures that reshape the cornea so that light traveling through the cornea is properly focused onto the retina located in the back of the eye. During LASIK eye surgery, an instrument called a microkeratome is used to cut a thin flap in the cornea. The cornea is then peeled back and the underlying cornea tissue ablated to the desired shape with an excimer laser. After the desired reshaping of the cornea is achieved, the cornea flap is put back in place and the surgery is complete.

The success of procedures, such as LASIK, in addressing eye disorders depends on the stability of the changes in the corneal structure after the procedures have been applied.

### BRIEF SUMMARY

Embodiments according to aspects of the present invention provide systems and methods for achieving corrective changes in corneal tissue and improving the stability of the changes to the corneal tissue. For example, a system for treating an eye includes a cutting instrument configured to make incisions in selected areas of the cornea; a cross-linking treatment system, the cross-linking treatment system including an applicator that applies a cross-linking agent to the selected areas of the cornea, a light source that provides photoactivating light for the cross-linking agent, and one or more optical elements that deliver the photoactivating light to the selected areas of the cornea, the photoactivating light acting on the cross-linking agent to initiate cross-linking activity in the selected areas of the cornea; and one or more controllers that determine the selected areas of the cornea for the incisions according to astigmatic keratotomy or radial keratotomy. Correspondingly, a method for treating an eye includes: selecting locations for making incisions in areas of the cornea according to astigmatic keratotomy or radial keratotomy; making, with a cutting instrument, incisions in the selected areas of the cornea; applying, with an applicator, a cross-linking agent to the selected areas of the cornea; and delivering, with one or more optical elements, photoactivating light from a light source to the selected areas of the cornea to initiate cross-linking activity in the selected areas of the cornea.

These and other aspects of the present disclosure will become more apparent from the following detailed description of embodiments of the present disclosure when viewed in conjunction with the accompanying drawings.

### BRIEF DESCRIPTION OF THE DRAWINGS

FIG. 1 provides a block diagram of an example delivery system for delivering a cross-linking agent and an activator to a cornea of an eye in order to initiate molecular cross-linking of corneal collagen within the cornea.

FIG. 2A illustrates a graph of depletion and gradual replenishment of oxygen below a 100 μm corneal flap saturated with 0.1% Riboflavin during 3 mW/cm² continuous wave (CW) irradiation.

FIG. 2B illustrates a graph of oxygen recovery under a 100 μm corneal flap saturated with 0.1% Riboflavin during 30 mW/cm² pulsed irradiation.

FIG. 3 illustrates a graph of absorbance of reduced Riboflavin before and after 30 mW/cm² CW irradiation for 3 minutes.

FIG. 4A illustrates a graph of fluorescence or Riboflavin samples at 450 nm.

FIG. 4B illustrates a graph of relative fluorescence of cross-linked Riboflavin flaps at different Riboflavin concentrations and depths.

FIGS. 5A-5C illustrate graphs of force versus displacement curves for porcine cornea for various soak times and UVA illumination scenarios.

FIG. 6 illustrates a graph of fluorescence versus wavelength for porcine cornea 200 μm flaps for various UVA illumination scenarios.

FIG. 7A illustrates a graph of cross-linking measured by fluorescence of the digested corneal flap at 450 nm for various UVA illumination scenarios.

FIG. 7B illustrates a graph of force versus displacement curve for porcine cornea for various UVA illumination scenarios.

FIG. 8 illustrates an example approach for stabilizing or strengthening corneal tissue by applying Riboflavin as a cross-linking agent according to aspects of the present invention.

FIG. 9 illustrates aspects of an eye anatomy.

FIG. 10 illustrates an example treatment that makes incisions to corneal tissue prior to an eye treatment that causes shape change in the cornea.

FIG. 11 illustrates an example system that makes incisions to corneal tissue prior to an eye treatment that causes shape change in the cornea.

FIG. 12 illustrates an example treatment that applies cross-linking treatments with intrastromal astigmatic keratotomy, according to aspects of the present invention.

FIG. 13 illustrates an example system that can be employed to apply cross-linking treatments with astigmatic keratotomy or radial keratotomy, according to aspects of the present invention.

While the invention is susceptible to various modifications and alternative forms, a specific embodiment thereof has been shown by way of example in the drawings and will herein be described in detail. It should be understood, however, that it is not intended to limit the invention to the particular forms disclosed, but on the contrary, the intention is to cover all modifications, equivalents, and alternatives falling within the spirit of the invention.

### DESCRIPTION

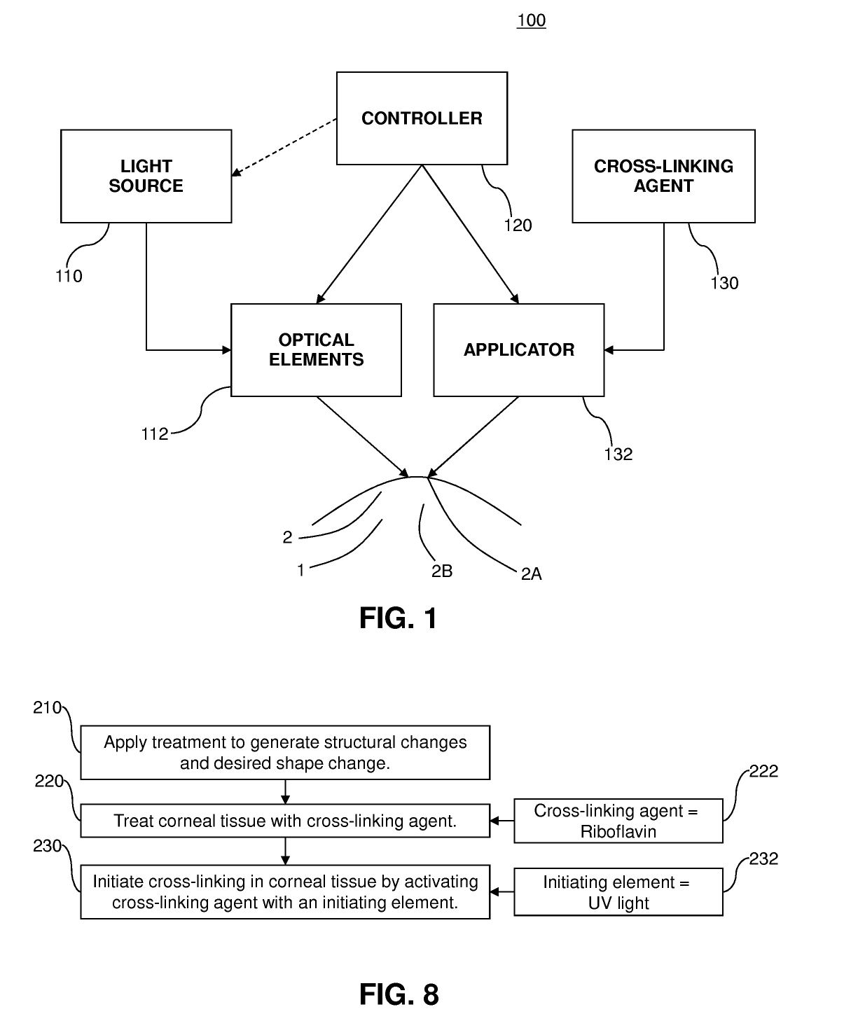

FIG. 1 illustrates an example delivery system 100 for delivering a cross-linking agent 130 to a cornea 2 of an eye 1 in order to initiate molecular cross-linking of corneal collagen within the cornea 2. The delivery system 100 includes an applicator 132 for applying the cross-linking agent 130 to the cornea 2. The delivery system 100 includes a light source 110 and optical elements 112 for directing light to the cornea 2. The delivery system 100 also includes a controller 120 that is coupled to the applicator 132 and the optical elements 112. The applicator 132 may be an apparatus adapted to apply the cross-linking agent 130 according to particular patterns on the cornea 2. The applicator 132 may apply the cross-linking agent 130 to a corneal surface 2A (e.g., an epithelium), or to other locations on the eye 1. Aspects for facilitating the delivery of the cross-linking agent 130 are described, for example, in U.S. patent application Ser. No. 14/062,467, filed Oct. 24, 2013, the contents of which are incorporated entirely herein by reference.

A large number of conditions and parameters affect the cross-linking of corneal collagen with the cross-linking agent. When the initiating element is ultraviolet-A (“UVA”) light, the irradiance and the dose both affect the amount and the rate of cross-linking. The UVA light may be applied continuously (continuous wave or CW) or as pulsed light, and this selection has an effect on the amount, the rate, and the extent of cross-linking. If the UVA light is applied as pulsed light, the duration of the exposure cycle, the dark cycle, and the ratio of the exposure cycle to the dark cycle duration all have an effect on both the rate of cross-linking and the amount of resulting corneal stiffening. Other factors that play a significant role in cross-linking include cross-linking agent concentration, temperature, specific conditions of the cornea (e.g., if any previous treatments have taken place), as well as other factors and parameters.

Aspects of the present disclosure relate to the effect of each of these parameters on the rate and the amount of cross-linking, as well as the interrelations of these parameters among each other to optimize the conditions to achieve the desired amount, rate, and location (on the cornea 2) of corneal stiffening. Aspects of the present disclosure relate to monitoring the corneal response to a change in one or a plurality of parameters and adjusting the one or the plurality of parameters based on the received feedback.

As described herein, the devices and approaches disclosed herein may be used to preserve desired shape or structural changes following an eye therapy treatment by stabilizing the corneal tissue of the cornea 2. The devices and approaches disclosed herein may also be used to enhance the strength or biomechanical structural integrity of the corneal tissue apart from any eye therapy treatment.

With reference to FIG. 1, the optical elements 112 may include one or more mirrors or lenses for directing and focusing the light emitted by the light source 110 to a particular pattern on the cornea 2 suitable for activating the cross-linking agent 130. The light source 110 may be a UVA light source that may also alternatively or additionally emit photons with greater or lesser energy levels than ultraviolet light photons. The delivery system 100 also includes a controller 120 for controlling the operation of the optical elements 112 or the applicator 132, or both. By controlling aspects of the operation of the optical elements 112 and the applicator 132, the controller 120 can control the regions of the cornea 2 that receive the cross-linking agent 130 and that are exposed to the light source 110. By controlling the regions of the cornea 2 that receive the cross-linking agent 130 and the light source 110, the controller 120 can control the particular regions of the cornea 2 that are strengthened and stabilized through cross-linking of the corneal collagen fibrils. In an implementation, the cross-linking agent 130 can be applied generally to the eye 1, without regard to a particular region of the cornea 2 requiring strengthening, but the light source 110 can be directed to a particular region of the cornea 2 requiring strengthening, to thereby control the region of the cornea 2 wherein cross-linking is initiated by controlling the regions of the cornea 2 that are exposed to the light source 110. Moreover, aspects of the present invention relate to modulating the specific regimes of the applied light to achieve a desired degree of corneal stiffening in selected regions of the cornea 2.

Another controller may be used to control the operation of the optical elements 112, and thereby control with precision the delivery of the light source 110 (i.e., the initiating element) to the cornea 2 by controlling any combination of: wavelength, bandwidth, intensity, power, location, depth of penetration, and duration (the duration of the exposure cycle, the dark cycle, and the ratio of the exposure cycle to the dark cycle duration) of treatment. In addition, the function of the controller 120 can be partially or wholly replaced by a manual operation.

Embodiments may also employ aspects of multiphoton excitation microscopy. In particular, rather than delivering a single photon of a particular wavelength to the cornea 2, the delivery system (e.g., 100 in FIG. 1) delivers multiple photons of longer wavelengths, i.e., lower energy, that combine to initiate the cross-linking. Advantageously, longer wavelengths are scattered within the cornea 2 to a lesser degree than shorter wavelengths, which allows longer wavelengths of light to penetrate the cornea 2 more efficiently than shorter wavelength light. Shielding effects of incident irradiation at deeper depths within the cornea are also reduced over conventional short wavelength illumination since the absorption of the light by the photosensitizer is much less at the longer wavelengths. This allows for enhanced control over depth specific cross-linking. For example, in some embodiments, two photons may be employed, where each photon carries approximately half the energy necessary to excite the molecules in the cross-linking agent 130 that release radicals (Riboflavin or photosensitizer and oxygen). When a cross-linking agent molecule simultaneously absorbs both photons, it absorbs enough energy to release reactive radicals in the corneal tissue. Embodiments may also utilize lower energy photons such that a cross-linking agent molecule must simultaneously absorb, for example, three, four, or five, photons to release a reactive radical. The probability of the near-simultaneous absorption of multiple photons is low, so a high flux of excitation photons may be required, and the high flux may be delivered through a femtosecond laser.

Aspects of the present disclosure, e.g., adjusting the parameters for delivery and activation of the cross-linking agent, can be employed to reduce the amount of time required to achieve the desired cross-linking. In an example implementation, the time can be reduced from minutes to seconds. While some configurations may apply the initiating element (i.e., the light source 110) at an irradiance of 5 mW/cm², aspects of the present disclosure allow larger irradiance of the initiating element, e.g., multiples of 5 mW/cm², to be applied to reduce the time required to achieve the desired cross-linking. Highly accelerated cross-linking is particularly possible when using laser scanning technologies in combination with a feedback system. The total dose of energy absorbed in the cornea 2 can be described as an effective dose, which is an amount of energy absorbed through an area of the corneal surface 2A. For example the effective dose for a region of the corneal surface 2A can be, for example, 5 J/cm², or as high as 20 J/cm² or 30 J/cm². The effective dose described can be delivered from a single application of energy, or from repeated applications of energy.

Aspects of the present disclosure provide systems and methods for delivering pulsed light of specific duty cycle and frequency, especially when a cross-linking agent is applied to stabilize desired shape changes generated in corneal tissue. Corneal cross-linking with Riboflavin is a technique that uses UVA light to photoactivate Riboflavin to stabilize and/or reduce corneal ectasia, in diseases such as keratoconus and post-LASIK ectasia. Corneal cross-linking improves corneal strength by creating additional chemical bonds within the corneal tissue.

According to aspects of the present disclosure, systems and methods generate pulsed light by employing a digital micro-mirror device (DMD), electronically turning a light source on and off, and/or using a mechanical or opto-electronic (e.g., Pockels cells) shutter or mechanical chopper or rotating aperture. DMD technology may be used to modulate the application of initiating light spatially as well as a temporally. Using DMD technology, a controlled light source projects the initiating light in a precise spatial pattern that is created by microscopically small mirrors laid out in a matrix on a semiconductor chip, known as a DMD. Each mirror represents one or more pixels in the pattern of projected light. The power and duration at which the light is projected is determined as described elsewhere. According to other aspects of the present disclosure, pulsed light may be generated in any suitable manner.

Riboflavin is deactivated (reversibly or irreversibly) and/or photo-degraded to a greater extent as irradiance increases. When Riboflavin absorbs radiant energy, especially light, it undergoes photosensitization. There are two major photochemical kinetic pathways for Riboflavin photosensitization, Type I and Type II. Some of the major reactions involved in both the Type I and Type II mechanisms are as follows:

Common Reactions for Type I and Type II Mechanisms

Rf→Rf*₁,I_{abs} (1)

Rf*₁→Rf,k1 (2)

Rf*₁→Rf*₃,k2 (3)

Type I Mechanism

Rf₃*+SH→(RF^{-}+SH^{+})→RfH^{}+S⁻,k3 (4)

2RfH^{}→Rf+RfH₂,k4 (5)

RfH₂+O₂→Rfₒₓ+H₂O₂,k5 (6)

### Type II Mechanism

Rf₃*+O₂→Rf+¹O₂,k6 (7)

SH+¹O₂→Sₒₓ,k6 (8)

Rf represents Riboflavin in the ground state. Rf*₁ represents Riboflavin in the excited singlet state. Rf*₃ represents Riboflavin in a triplet excited state. Rf^{-} is the reduced radical anion form of Riboflavin. RfH^{} is the radical form of Riboflavin. RfH₂ is the reduced form of Riboflavin. SH is the substrate. SW is the intermediate radical cation. S^{} is the radical. Sₒₓ is the oxidized form of the substrate. Rfₒₓ is deuteroflavin (7,8-dimethyl-10-(formylmethyl)isoalloxazine) having UVA absorption and sensitizer properties similar to those of Riboflavin (and unlike those of RfH₂).

Riboflavin is excited into its triplet excited state Rf*₃ as shown in reactions (1) to (3). From the triplet excited state Rf*₃, the Riboflavin reacts further, generally according to Type I or Type II photomechanical mechanisms.

Type I mechanism above is favored at low oxygen concentrations, and Type II mechanism is favored at high oxygen concentrations. In Type I mechanism, the substrate reacts with the sensitizer excited state to generate radicals or radical ions, respectively, by hydrogen atoms or electron transfer. In Type II mechanism, the excited sensitizer reacts with oxygen to form singlet molecular oxygen. The singlet molecular oxygen then acts on tissue to produce additional cross-linked bonds.

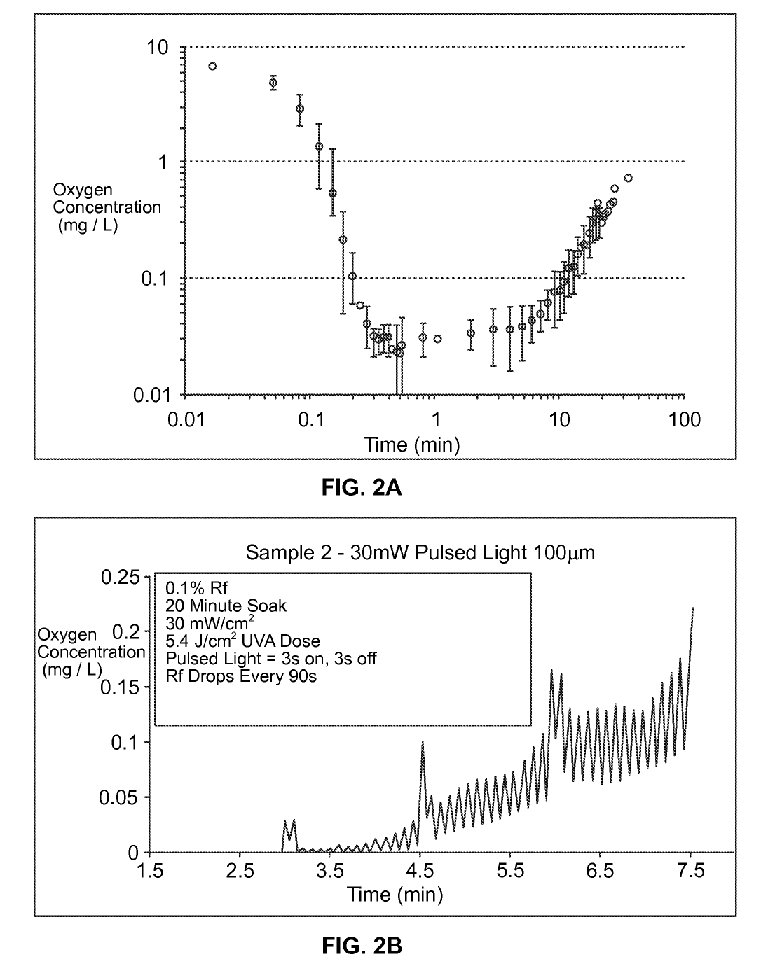

Oxygen concentration in the cornea is modulated by UVA irradiance and temperature and quickly decreases at the beginning of UVA exposure. The oxygen concentration tends to deplete within about 10-15 seconds for irradiance of 3 mW/cm² (as shown, for example, in FIG. 2A) and within about 3-5 seconds for irradiance of 30 mW/cm². Utilizing pulsed light of a specific duty cycle, frequency, and irradiance, input from both Type I and Type II photochemical kinetic mechanisms may be optimized to achieve the greatest amount of photochemical efficiency. Moreover, utilizing pulsed light allows regulating the rate of reactions involving Riboflavin. The rate of reactions may either be increased or decreased, as needed, by regulating, one of the parameters such as the irradiance, the dose, the on/off duty cycle, Riboflavin concentration, soak time, and others. Moreover, additional ingredients that affect the reaction and cross-linking rates may be added to the cornea.

One aspect of the present disclosure relates to achieving photon optimization by allowing deactivated (reduced) Riboflavin to return to ground state Riboflavin in Type I reactions and allowing for reduced rate of oxygen uptake in Type II reactions where better photon conversion efficiency occurs.

The rate of return of deactivated (reduced) Riboflavin to ground state in Type I reactions and the rate of oxygen uptake in Type II reactions is determined by a number of factors. These factors include, but are not limited to, on/off duty cycle of pulsed light treatment, pulse rate frequency, irradiance, and dose. Moreover, the Riboflavin concentration, soak time, and addition of other agents, including oxidizers, affect the rate of oxygen uptake. These and other parameters, including duty cycle, pulse rate frequency, irradiance, and dose are optimized to achieve optimal photon efficiency and make efficient use of both Type I and Type II photochemical kinetic mechanisms for Riboflavin photosensitization. Moreover, these parameters are optimized in such a way as to achieve an optimum chemical amplification effect.

According to aspects of the present disclosure, for pulsed light treatment, the on/off duty cycle is between approximately 100/1 to approximately 1/100; the irradiance is between approximately 1 mW/cm² to approximately 500 mW/cm² average irradiance, and the pulse rate is between approximately 0.1 Hz to approximately 1000 Hz.

According to aspects of the present disclosure, for pulsed light treatment, the on/off duty cycle is between approximately 1000/1 to approximately 1/1000; the irradiance is between approximately 1 mW/cm² to approximately 1000 mW/cm² average irradiance, and the pulse rate is between approximately 1000 Hz to approximately 100,000 Hz. These averages are based on using a q-switched laser source instead of an LED system where higher repetition rates are possible. According to further aspects of the present disclosure, the laser source may be an adjustable pulsed source, an LED system, arc sources or incandescents at very long on-time duty cycles, or any other suitable sources.

Pulse rates of 0.1 Hz to approximately 1000 Hz or 1000 Hz to approximately 100,000 Hz may be chosen based on the photochemical kinetics as detailed by Kamaev et al., Investigative Ophthalmology & Visual Science, April 2012, Vol. 53, No. 4, pp. 2360-2367 (April 2012), which is incorporated herein by reference in its entirety. According to aspects of the present disclosure, the pulse length may be long—on the order of one or several seconds—or short—on the order of fractions of a second.

According to aspects of the photochemical kinetics of corneal cross-linking with Riboflavin, pulsed light illumination can be used to create greater or lesser stiffening of corneal tissue than may be achieved with continuous wave illumination for the same amount or dose of energy delivered. Light pulses of suitable length and frequency may be used to achieve optimum chemical amplification.

FIG. 2A illustrates a graph of depletion and gradual replenishment curve of dissolved oxygen below a 100 μm thick porcine corneal flap, saturated with 0.1% Riboflavin during 3 mW/cm² UVA irradiation at 25° C. The oxygen concentration (mg/L) fell to zero at about 15 seconds and gradually started to increase after approximately 10 minutes, getting back to approximately one-tenth its starting value after 30 minutes.

FIG. 2B illustrates a graph of oxygen recovery under a 100 μm thick corneal flap. The corneal flap was saturated with 0.1% Riboflavin during 30 mW/cm² UVA irradiation. The irradiation was pulsed at a 3 second on/3 seconds off cycle. Riboflavin drops were added to the cornea every 90 seconds. In this example, it took about 3 minutes for the oxygen concentration to gradually start increasing and about 6 minutes for the oxygen concentration to increase to 0.1 mg/L.

Under aerobic conditions, which are present during the first 10 to 15 seconds of UVA exposure, sensitized photo-oxidation of the substrate (proteoglycan core proteins and collagen in the corneal matrix) occurs mainly by its reaction with photochemically generated reactive oxygen species, such as singlet molecular oxygen. This is consistent with a Type II photochemical mechanism. After the first 10 to 15 seconds, oxygen becomes totally depleted and the reaction between the substrate and Riboflavin becomes consistent with a predominantly Type I photochemical mechanism. More than halfway through the period of illumination, the oxygen concentration in the cornea slowly increases to a concentration at which a Type II mechanism may begin to play an additional role. During this phase, a growing contribution would be expected from the singlet oxygen-mediated cross-linking, together with the enhancement of secondary radical reactions that are modulated by oxygen.

Studies of corneal flaps taken at various depths after cross-linking, and tested by stress-strain behavior or collagen fluorescence analysis, suggest that corneal stiffening is primarily in the anterior 200 μm of the corneal stroma. Increase of collagen fluorescence in UVA-exposed corneas, which is related to their mechanical stiffening, can be detected at a depth 200 to 300 μm from the corneal surface as will be described further below.

If UVA radiation is stopped shortly after oxygen depletion, oxygen concentrations start to increase (replenish) as shown in FIGS. 2A and 2B. Excess oxygen may be detrimental in corneal cross-linking process because oxygen is able to inhibit free radical photopolymerization reactions by interacting with radical species to form chain-terminating peroxide molecules. The pulse rate, irradiance, dose, and other parameters may be adjusted to achieve an optimized oxygen regeneration rate. Calculating and adjusting the oxygen regeneration rate is another example of adjusting the reaction parameters to achieve a desired amount of corneal stiffening.

Dissolved free oxygen is significantly depleted not only at the position of the oxygen sensor and below, but also throughout the corneal flap above. Oxygen content may be depleted throughout the cornea, by various chemical reactions, except for the very thin corneal layer where oxygen diffusion is able to keep up with the kinetics of the reactions. This diffusion-controlled zone will gradually move deeper into the cornea as the reaction ability of the substrate to uptake oxygen decreases.

Oxygen measurements in the cornea suggest that the predominant photosensitizing mechanism for cross-linking with Riboflavin is the Type I pathway after a very short initial Type II photochemical mechanism at the start of the illumination with UVA light. More than halfway through the period of illumination, the oxygen concentration in the cornea slowly increases, as shown in FIGS. 2A and 2B above, to a concentration at which a Type II mechanism may begin to play an additional role.

The mechanism for corneal cross-linking begins with the additional pathway kinetics expressed in equation (6) above. After a short period of time (a few seconds), oxygen becomes depleted, and there is little oxygen available as shown in FIG. 2A. Under these anaerobic conditions, Leuco-Deuteroflavin+H₂O₂ are formed as described in Heldman et al., Handbook of Food Engineering (2^{nd} Edition), CRC Press (2006). Leuco-Deuteroflavin has low absorption at 360 nm and lacks the photosensitizing ability and therefore cannot create radicals. Leuco-Deuteroflavin is referred to herein as reduced flavin, reduced Riboflavin, RfH₂, and Fl_{red}H₂.

Reduced Riboflavin undergoes an oxidation reaction as shown in equation (6) above. The oxidation of reduced Riboflavin by molecular oxygen is irreversible, autocatalytic, and involves generation of free radicals that can initiate radical polymerization (as in case of vinyl monomers, acrylamide with bis(acrylamide), etc.). The autocatalytic oxidation of reduced Riboflavins by oxygen is accounted for by the reactions described in Massey, V., Activation of molecular oxygen by flavins and flavoproteins, J. Biol. Chem. (1994), 269, 22459-22462.

Depending on the particular circumstances, there may be a need to either speed up or slow down the oxidation rate of the reduced Riboflavin. A number of parameters affect the reaction rate and cross-linking rate of the reduced Riboflavin.

During pulsed light irradiation, when the UVA light is turned off (or turned down to a lower value), following the many pathways described by Massey, oxygen is regenerated locally near the Leuco-Deuteroflavin converting it into Deuteroflavin which is able to absorb light again and thereby create radicals for cross-linking. Therefore, improved photon efficiency is achieved through the proper timing of on/off cycles through the regeneration of Deuteroflavin. Regeneration of Deuteroflavin allows for larger overall concentration of radical generation for a given light energy dose than under continuous wave illumination and continuous anaerobic conditions.

Oxygen is the naturally occurring oxidizer and is used as the oxidizer according to aspects of the present disclosure. According to further aspects of the present disclosure, oxygen and/or other oxidizers are utilized; such oxidizers may be added to the formulation or administered to the cornea in a suitable way.

According to other embodiments of the present disclosure, reduced Riboflavin may be soaked in a suitable agent that contains oxygen and is able to oxidize the reduced Riboflavin. According to one embodiment, Vitamin B12 may be added in any suitable manner to the reduced Riboflavin and/or to the cornea. Vitamin B12 contains a Cobalt molecule that is capable of holding oxygen, thereby creating an oxygen storage reservoir. The reduced Riboflavin may be super-saturated with Vitamin B12 or another suitable oxygen carrying agent. The suitable agent, such as Vitamin B12, may be provided in conjunction with application of pulsed light. The proper level of oxygen can be maintained with various reversible oxygen carriers. See Yang N., Oster G. Dye-sensitized photopolymerization in the presence of reversible oxygen carriers. J. Phys. Chem. 74, 856-860 (1970), the contents of which are incorporated entirely herein by reference.

Corneal stiffening may be applied to the cornea according to particular patterns, including, but not limited to, circular or annular patterns, which may cause aspects of the cornea to flatten and improve vision in the eye. For example, more or less corneal stiffening may be desired on the outer edges of the cornea as opposed to the center of the cornea. Aspects of the present disclosure relate to achieving more corneal stiffening on the outer diameter of the cornea and gradually decreasing the amount of corneal stiffening from the outer diameter toward the center of the cornea. Other aspects of the present disclosure relate to selecting regions of the cornea that require more corneal stiffening based on a predetermined set of characteristics and applying more corneal stiffening to those selected regions by varying the regime of the pulsed light. According to certain aspect of the present disclosure, pulsed light may be applied with different irradiance, dose and/or different duty cycle to different areas of the cornea, leading to areas of differing levels of corneal stiffening or corneal stiffening gradients. Varying the regime of the pulsed light to achieve a desired level of corneal stiffening is another example of adjusting the parameters of the cross-linking reaction to achieve specific, targeted results. Moreover, varying the regime of the pulsed light to achieve a desired level of corneal stiffening at selected regions of the cornea allows for more precise and accurate control of the shape changes in the eye.

Approaches for applying the initiating element such as UV light according to a selected pattern are described, for example, in U.S. patent application Ser. No. 13/438,705, filed Apr. 3, 2012, and U.S. patent application Ser. No. 13/051,699, filed Mar. 18, 2011, the contents of these applications being incorporated entirely herein by reference.

Currently, at least three different paths leading to cross-links may be utilized: a path through the direct attack of the excited Riboflavin's triplets (Type I mechanism), through singlet oxygen (Type II), and through the generation of free radicals when the reduced Riboflavin (RFH₂) interacts with oxygen (as seen in the scheme below).

The reduced form of Riboflavin RFH₂ (with two hydrogen atoms supplied to the aromatic nucleus by the side chain) can be produced by anaerobic photolysis of Riboflavin (Holmstrom 1961) and observed by the reduction in absorption at 445 nm.

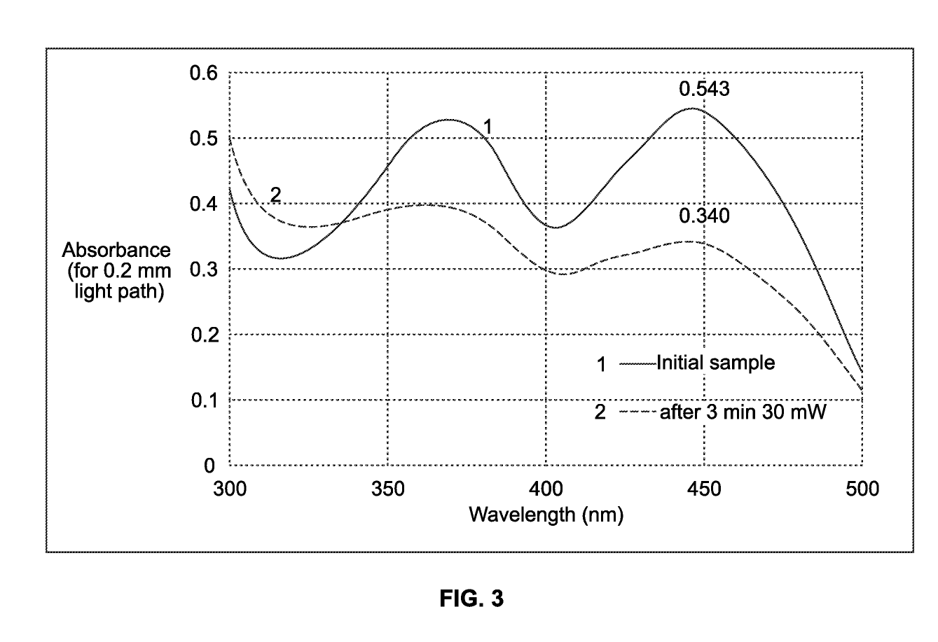

Referring now to FIG. 3, a graph illustrating absorbance for 0.2 mm light path of an initial sample of Riboflavin versus absorbance for 0.2 mm light path of reduced Riboflavin RfH₂ after being irradiated for 3 minutes at an irradiance of 30 mW/cm². At a wavelength of 445 nm, the absorbance of the initial sample is about 0.543, while the absorbance of the irradiated sample is about 0.340.

RFH₂ is autoxidizable and in the presence of oxygen yields the highly light-sensitive fluorescent and absorbing (445 nm) Deuteroflavin (7,8-dimethyl-10-(formylmethyl)isoalloxazine). Reduced Riboflavin solutions can be prepared under nitrogen by irradiation of Riboflavin with visible light in the presence of EDTA and stored in absence of oxygen. Reaction with oxygen completes during hundreds of msec (depending on the initial conditions), proceeds via free radicals (as described in Massey), and is able to initiate polymerization of vinyl monomers. The rate of the reaction with oxygen may be increased by dissolving oxygen in a flavin solution instead of in water.

### Example 1

### Fluorescence of Corneal Samples

### Materials and Methods

RFH₂ was prepared by irradiation of Riboflavin solutions (with and without EDTA, 1% EDTA, 0.1% Riboflavin) and saturated with argon (to displace oxygen) in a shallow sealed quartz cuvette. Then, in the absence of additional UV light, porcine corneal flaps were immediately placed in those solutions. After 1-2 min this procedure was repeated with a fresh solution containing RFH₂ several times. Corneal flaps then were washed with distilled water, digested with papain buffer, and their fluorescence was measured.

### Results

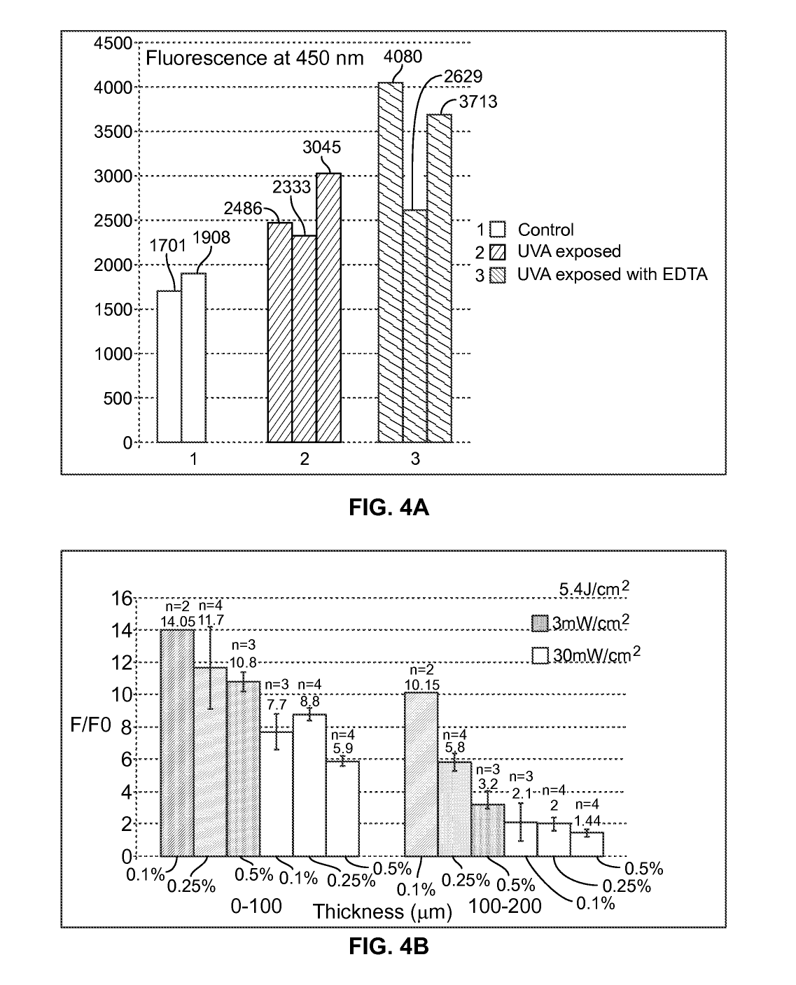

As seen in FIG. 4A, fluorescence of the corneal samples treated with preliminary UVA-exposed Riboflavin solutions, was higher than fluorescence of the corneal samples washed with pure, unexposed Riboflavin. This can indicate that oxidation of RFH₂ is able to generate some cross-linking in the cornea, but observed fluorescence was significantly lower than generally observed in corneal flaps directly exposed to the UVA. The fluorescence was the highest for the sample prepared by irradiation of Riboflavin solutions with a reducing agent such as EDTA. Accordingly, one way of increasing the cross-linking efficiency of RfH₂ is to use Riboflavin in a solution with a reducing agent for Riboflavin. The reducing agents may include, but are not limited to, EDTA, ascorbic acid, sugars, amines, amino acids, and any combination thereof. This is another example of modifying the parameters of the cross-linking reaction to achieve a desired level of cross-linking with the corneal fibrils. According to one aspect of the present invention, Riboflavin concentrations between about 0.001% Riboflavin to about 1.0% Riboflavin may be utilized.

It was found that fluorescence intensity of collagen (as in the original material or in the material digested by enzymes like papain solution) linearly correlates with its stiffness. For example, this correlation was reported for corneal collagen in Chai et al. Quantitative Assessment of UVA-Riboflavin Corneal Cross-Linking Using Nonlinear Optical Microscopy. Investigative Opthalmology & Visual Science, June 2011, Vol. 52, No. 7 (2011), the contents of which are incorporated entirely herein by reference. This correlation was reported for collagen fibers from Wistar rats in Rolandi et al. Correlation of Collagen-Linked Fluorescence and Tendon Fiber Breaking Time. Gerontology 1991; 27:240-243, the contents of which are incorporated entirely herein by reference. This correlation was reported for collagen in chondrogetic samples in Fite et al. Noninvasive Multimodal Evaluation of Bioengineered Cartilage Constructs Combining Time-Resolved Fluorescence and Ultrasound Imaging. Tissue Eng: Part C Vol. 17, Number 4, 2011, the contents of which are incorporated entirely herein by reference. This correlation was reported for collagen network in human articular cartilage in Verzijl et al. Crosslinking by Advanced Glycation End Products Increases the Stiffness of the Collagen Network in Human Articular Cartilage. Arthritis & Rheumatism Vol. 46, No. 1, January 2002, pp. 114-123, the contents of which are incorporated entirely herein by reference.

Cross-linking efficacy tends to decrease at higher Riboflavin concentrations. See Song P., Metzler D. Photochemical Degradation of Flavins-IV. Studies of the Anaerobic Photolysis of Riboflavin. Photochemistry and Photobiology, Vol. 6, pp. 691-709, 1967, the contents of which are incorporated entirely herein by reference (explaining the shielding effect of Riboflavin and that quenching of the triplet state by Riboflavin itself (concentration quenching) must be considered, and that the products of photochemical cleavage decrease the quantum yield rapidly as they accumulate.)

### Example 2

### Measurement of the Collagen Linked Fluorescence in Cross-Linked Corneal Flaps at Depths of 100 μm and 200 μm

### Materials and Methods

Porcine whole globes (SiouxPreme Packing Co., Sioux City, Iowa; shipped in saline solution packed in ice) were warmed to room temperature (25° C.). The corneas were then de-epithelialized with a dulled scalpel blade and 0.1, 0.25, or 0.5% riboflavin solution in 0.9% saline was applied to the top of each cornea during 20 minutes before cross-linking. Corneas were pan-corneally irradiated with a top hat beam (3% root mean square) for a determined amount of time with 365-nm light source (UV LED NCSU033B[T]; Nichia Co., Tokushima, Japan) at the chosen irradiance (3 or 30 mW/cm²) which was measured with a power sensor (model PD-300-UV; Ophir, Inc., Jerusalem, Israel) at the corneal surface.

Corneal flaps (each 100 μm thick, one after another) were excised from the eyes with aid of Intralase femtosecond laser (Abbott Medical Optics, Santa Ana, Calif.). The average thickness of the corneal flaps was calculated as a difference between the measurements before and after the excision from the eyes with an ultrasonic Pachymeter (DGH Technology, Exton, Pa.). The flaps were washed with distilled water until Riboflavin in the washing waters was not detectable by absorbance measurement at 455 nm (Thermo Scientific Evolution 300/600 UV-Vis Spectrophotometer, Thermo Fisher Scientific, Waltham, Mass.). The flaps then were dried in vacuum until the weight change became less than 10% (Rotary vane vacuum pump RV3 A652-01-903, BOC Edwards, West Sussex, UK).

Each flap (1 mg) was digested for 2 h at 65° C. with 2.5 units/ml of papain (from Papaya latex, Sigma) in 0.5 ml of papain buffer [1×PBS (pH 7.4), 2 mM L-cysteine and 2 mM EDTA]. Papain digests were centrifuged for 30 seconds at 2200×G (Mini centrifuge 05-090-100, Fisher Scientific), diluted 3 times with 1×PBS solution and fluorescence of the solutions was measured with excitation of λex=360 nm in a QM-40 Spectrofluorometer (Photon Technology Int., London, Ontario, Canada). The fluorescence of the papain buffer was taken into account by measuring fluorescence in the absence of tissue and subtracting this value from the fluorescence of the samples.

### Results

Fluorescence of the excised samples, which was a relative value to the non-cross-linked samples with the same thickness, is illustrated in FIG. 4B. It is shown that the corneal fluorescence after cross-linking (all samples were exposed to the same UVA dose, 5.4 J/cm²) is greater for samples exposed to UVA for a longer duration with lower irradiance and lower concentration of Riboflavin. Corneal fluorescence is also greater at first 100 μm than at the next 100 μm in the cornea. The highest corneal fluorescence was observed at the first 100 μm for the sample that was soaked in 0.1% Riboflavin solution and irradiated at 3 mW/cm².

### Example 3

### Measurement of Biomedical Stiffness

### Materials and Methods

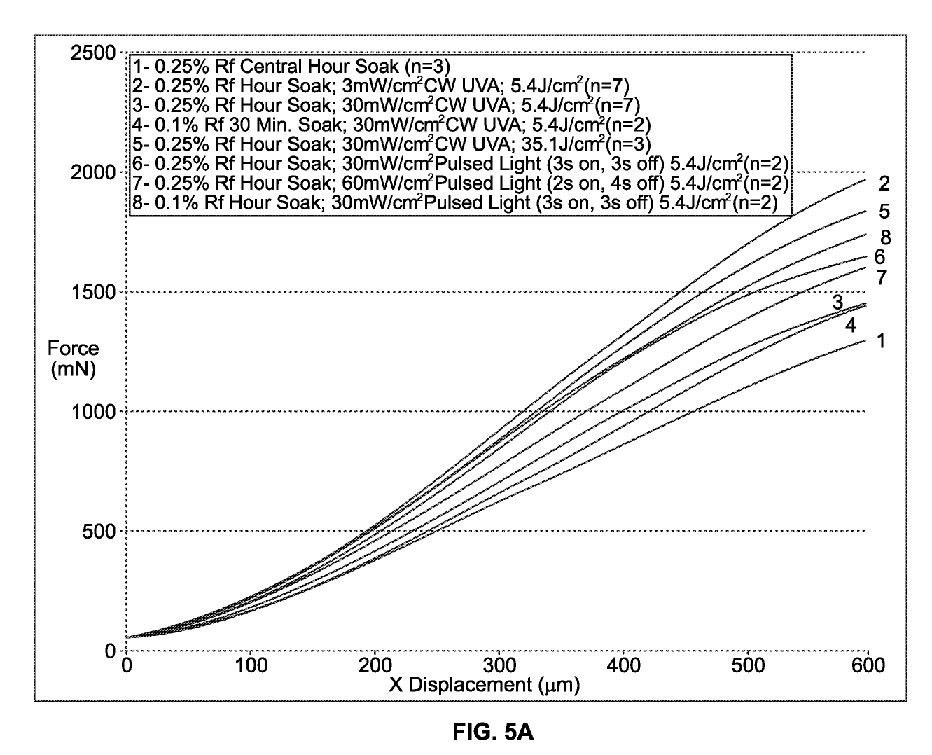

Upon arrival, porcine eyes have excess muscle tissue that was removed and placed in saline in the incubator (set to 37° C.) for 30 minutes. Eyes were then de-epithelialized and placed in a 0.1% Riboflavin solution for 20 minutes at 37° C. Eyes were removed from solution and physiological IOP was applied. Eyes were then placed under a UVA source and shutter system and irradiated according to the indicated protocol as shown in FIGS. 5A-5C. Riboflavin drops were applied every 1.5 minutes during UVA application. After being irradiated, the corneal thickness was measured with a pachymeter. The sample was then placed under the femto second laser and a ˜200 μm flap was cut. The flap was positioned in a biaxial materials tester (CS-BIO TESTER 5000, CellScale, Waterloo, ON Canada) and stretched until failure. The sample was then rinsed with distilled water and frozen for future papain digestion and fluorescence analysis.

### Results

FIG. 5A illustrates force versus displacement curves for porcine cornea for various soak times and UVA illumination scenarios. FIG. 5A illustrates results of experiments that show dissimilar biomechanical stiffness of the 0.25% Riboflavin sample 2 irradiated with 3 mW/cm² continuous wave illumination vs. the sample 3 irradiated with 30 mW/cm² continuous wave illumination for a total 5.4 J/cm² dose delivered. The biomechanical stiffness of the 0.25% Riboflavin sample 3 irradiated with 30 mW/cm² continuous for a total 5.4 J/cm² dose delivered was similar to the biomechanical stiffness of the 0.1% Riboflavin sample 4 irradiated under the same conditions. The biomechanical stiffness of the 0.25% Riboflavin sample 6 irradiated with 30 mW/cm² pulsed light with a 3 seconds on/3 seconds off duty cycle was similar to the biomechanical stiffness of 0.25% Riboflavin sample 7 irradiated with 60 mW/cm² pulsed light with a 2 seconds on/4 seconds off duty cycle for a total 5.4 J/cm² dose delivered to each sample. The biomechanical stiffness of the 0.1% Riboflavin sample 8 irradiated with 30 mW/cm² pulsed light with a 3 seconds on/3 seconds off duty cycle was higher than both the samples 6 and 7. The biomechanical stiffness of the Riboflavin sample 5 irradiated with 30 mW/cm² continuous wave illumination for a total 35.1 J/cm² dose delivered was higher than that for samples 6, 7, 3, 4, and 1 (control sample with 0.25% Riboflavin concentration) and lower than that for sample 2. Sample 4 was soaked with Riboflavin for 30 minutes, while all the other samples were soaked with Riboflavin for 1 hour.

FIG. 5A illustrates the effect of varying different parameters on corneal cross-linking. Different parameters—irradiance, continuous wave vs. pulsed illumination, on/off duty cycle of pulsed light illumination, Riboflavin concentration, and other parameters—all have an effect on biomechanical stiffness.

A DMD may be used for illumination. With the DMD one can perform topography guided cross-linking as described, for example, in U.S. patent application Ser. No. 13/438,705, filed Apr. 3, 2012, and U.S. patent application Ser. No. 13/051,699, filed Mar. 18, 2011, the contents of which are incorporated entirely herein by reference. The algorithms associated with the topography may be created using several different spatial and temporal irradiance and dose profiles.

These spatial and temporal dose profiles may be created using continuous wave illumination but may also be modulated via pulsed illumination by pulsing the illumination source under varying frequency and duty cycle regimes as described above. Or, the DMD may be able to modulate different frequencies and duty cycles on a pixel by pixel basis to give ultimate flexibility using continuous wave illumination. In a third alternative, both pulsed illumination and modulated DMD frequency and duty cycle combinations may be combined. This allows for specific amounts of spatially determined corneal cross-linking. This spatially determined cross-linking may be combined with dosimetry, interferometry, optical coherence tomography (OCT), corneal topography, etc., for real-time modulated corneal cross-linking. Additionally, the pre-clinical patient information may be combined with finite element biomechanical computer modeling to create patient specific pre-treatment plans.

Because of the pixel specific modulation capabilities of the DMD and the subsequent stiffness impartment based on the modulated frequency, duty cycle, irradiance and dose delivered to the cornea, complex biomechanical stiffness patterns may be imparted to the cornea to allow for various amounts of refractive correction. These refractive corrections may include combinations of myopia, hyperopia, astigmatism, irregular astigmatism, presbyopia and complex corneal refractive surface corrections because of ophthalmic conditions such as keratoconus, pellucid marginal disease, post-lasik ectasia, and other conditions of corneal biomechanical alteration/degeneration, etc.

A specific advantage of the DMD system and method is that it allows for randomized asynchronous pulsed topographic patterning, creating a non-periodic and uniformly appearing illumination which eliminates the possibility for triggering photosensitive epileptic seizures or flicker vertigo for pulsed frequencies between 2 Hz and 84 Hz as described above.

Thus, applying pulsed light instead of continuous wave illumination has an effect on biomechanical stiffness. This is yet one more parameter that may be altered in optimizing cross-linking.

Flicker vertigo, sometimes called the Bucha effect, is an imbalance in brain-cell activity caused by exposure to low-frequency flickering (or flashing) of a relatively bright light. It is a disorientation-, vertigo-, and nausea-inducing effect of a strobe light flashing at 1 Hz to 20 Hz, which corresponds approximately to the frequency of human brainwaves. The effects are similar to seizures caused by epilepsy (particularly, photosensitive epilepsy), but are not restricted to people with histories of epilepsy. In the United States, websites provided by federal agencies are governed by section 508 of the Rehabilitation Act. The Act says that pages shall be designed to avoid causing the screen to flicker with a frequency between 2 Hz and 55 Hz. The 508 regulations are currently being updated and are expected to use the same criteria as WCAG 2.0 when completed. (Section 508 Subpart B—Technical Standards §1194.21 Software applications and operating systems.—(k) Software shall not use flashing or blinking text, objects, or other elements having a flash or blink frequency between 2 Hz and 55 Hz.) Photosensitive seizures are those triggered by either flashing or flickering lights, or rapidly changing geometric shapes or patterns. Many people with epilepsy are unaware that they are sensitive to certain kinds of lights or flickering patterns until they have a seizure. Less than 5% of those who suffer from epilepsy are photosensitive. This means that approximately one in 4,000 individuals suffer from this—which corresponds to less than 100,000 in the U.S. population. The characteristics of each individual's susceptibility are unique. A certain photosensitive individual may not be susceptible to a given light display at all. Still it is clear that every public display of lights can expect to regularly affect photosensitive epileptics—thus, a high degree of diligence is due the effort to eliminate displays which may trigger seizures. It is well documented that the range of 15 to 20 Hz is of greatest concern, however some individuals are susceptible to flashing lights as slow as 5 Hz and some as high as 84 Hz.

FIG. 5B illustrates force versus displacement curves for samples of porcine cornea irradiated with pulsed light having various exposure times, as well as a curve for a sample irradiated with continuous wave illumination, and a curve for a control sample. All the samples that were irradiated with pulsed light had a 0.1% Riboflavin concentration and were irradiated with 30 mW/cm² pulsed light with a 3 seconds off cycle and a varied exposure (“on”) cycle. The sample 2 having a 4.5 second exposure cycles had slightly lower biomechanical stiffness than samples 3 or 1. Thus, the duration of the exposure cycle affects the amount of corneal stiffening at the same irradiance and dark phase duration. Therefore, aspects of the present disclosure affect the displacement per unit force ratio. This is yet one more parameter that may be altered in optimizing cross-linking.

FIG. 5C illustrates force versus displacement curves for samples of porcine cornea illuminated with pulsed light having varied dark phase durations, as well as curves for samples irradiated with continuous illumination, and a curve for a control sample. The sample 1 having 0.1% Riboflavin concentration irradiated with 3 mW/cm² continuous wave illumination had the highest biomechanical stiffness. The samples having 0.1% Riboflavin concentration that were irradiated with 30 mW/cm² pulsed light with 3 seconds on/4.5 seconds off (sample 2) and with 3 seconds on/3 seconds off (sample 4) duty cycles had slightly lower biomechanical stiffness, followed by, in descending order, samples irradiated with 30 mW/cm² pulsed light with 3 seconds on cycles and 1.5 (sample 5), 0.75 (sample 6), and 0.25 (sample 7) seconds off (dark phase) cycles. The sample 2 having 0.25% Riboflavin concentration irradiated with 3 mW/cm² continuous wave illumination had the lowest biomechanical stiffness out of samples 1 and 3-7. Thus, the duration of the dark phase affects the amount of corneal stiffening even at the same irradiance and exposure duration. This is yet one more parameter that may be altered in optimizing cross-linking.

The graphs in FIGS. 5A-5C show that varying different parameters—applying pulsed instead of continuous wave illumination, varying on/off duty cycles, irradiance, dose, Riboflavin concentration, and soak times—all have an effect on biomechanical stiffness. These parameters may be modified in such a way as to achieve an optimum or desired amount of corneal stiffness anywhere on or within the cornea.

In a corroborating experiment, porcine eyes were de-epithelialized and placed in the 0.1% Riboflavin solution for 20 minutes. Eyes were removed from solution and physiological IOP was applied. A flap was cut using a femto second laser and an O₂ sensor was placed under the flap. UVA illumination was administered as indicated and Riboflavin drops were applied every 90 seconds for the duration of the dose delivered. After cross-linking the flap using the pulsed light dosing, the flap was removed and tested mechanically as described above.

Results from the O₂ sensor (FIG. 2B) show that under pulsed light irradiation, oxygen slightly increases cyclically with the duty cycle of the pulsed light. It appears that locally available H₂O₂ during the dark phase of the pulsed light cycle does indeed supply small amounts of oxygen back into the system capable of allowing reactivation of reduced Riboflavin and its conversion into Deuteroflavin (Rfₒₓ) as described above. Oxygen diffusion from the surface by itself takes much longer to account for these observations.

### Example 4

### Fluorescence of Samples Irradiated with Continuous Wave Illumination Versus Pulsed Light Illumination

### Materials and Methods

In another corroborating experiment porcine whole globes (SiouxPreme Packing Co., Sioux City, Iowa; shipped in saline solution packed in ice) were warmed to room temperature (25 C). The corneas were then de-epithelialized with a dulled scalpel blade and 0.1, Riboflavin solution in 0.9% saline was applied to the top of each cornea during 20 minutes before cross-linking in an incubator. Corneas were pan-corneally irradiated with a top hat beam (3% root mean square) for a determined amount of time with 365-nm light source (UV LED NCSU033B[T]; Nichia Co., Tokushima, Japan) at the chosen irradiance of 30 mW/cm² with either continuous wave illumination or pulsed illumination 3 seconds on/3 Seconds off. Corneal flaps (200 μm thick) were excised from the eyes with aid of Intralase femtosecond laser (Abbott Medical Optics, Santa Ana, Calif.). The average thickness of the corneal flaps was calculated as a difference between the measurements before and after the excision from the eyes with an ultrasonic Pachymeter (DGH Technology, Exton, Pa.).

The flaps were washed with distilled water until Riboflavin in the washing waters was not detectable by absorbance measurement at 455 nm (Thermo Scientific Evolution 300/600 UV-Vis Spectrophotometer, Thermo Fisher Scientific, Waltham, Mass.). The flaps then were dried in vacuum until the weight change became less than 10% (Rotary vane vacuum pump RV3 A652-01-903, BOC Edwards, West Sussex, UK). Each flap (1 mg) was digested for 2 h at 65° C. with 2.5 units/ml of papain (from Papaya latex, Sigma) in 0.5 ml of papain buffer [1×PBS (pH 7.4), 2 mM L-cysteine and 2 mM EDTA]. Papain digests were centrifuged for 30 seconds at 2200×G (Mini centrifuge 05-090-100, Fisher Scientific), diluted 3 times with 1×PBS solution and fluorescence of the solutions was measured with excitation of λex=360 nm in a QM-40 Spectrofluorometer (Photon Technology Int., London, Ontario, Canada).

The fluorescence of the papain buffer was taken into account by measuring fluorescence in the absence of tissue and subtracting this value from the fluorescence of the samples. The results seen below corroborate that the amount of fluorescence seen which is directly related to the amount of cross-linking is greater for the pulsed vs. continuous wave illumination in nearly the same proportion as the biomechanical measurements taken of the same flaps.

### Results

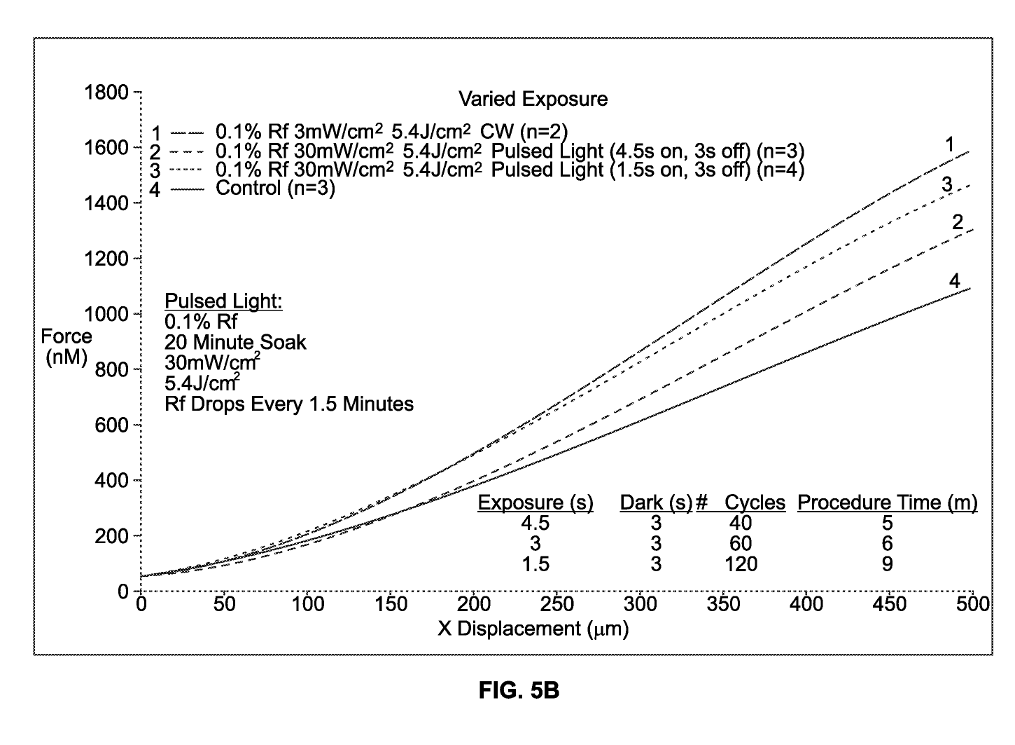

Referring now to FIG. 6, a graph of fluorescence versus wavelength curves is shown. At 455 nm, fluorescence was the highest—about 20,000 counts/s—for the sample irradiated with 30 mW/cm² pulsed light illumination with a 3 seconds on/3 seconds off cycle. The fluorescence for the sample irradiated with 30 mW/cm² continuous illumination was about 40% less, or about 12000 counts/s. This is yet another example demonstrating that applying pulsed light illumination as opposed to continuous wave illumination affects cross-linking in the cornea.

### Example 5

### Cross-Linking Measured by Fluorescence of Digested Corneal Flaps at 450 nm

### Materials and Methods

Pig eyes from an abattoir (SiouxPreme, Sioux City, Iowa) were rinsed in saline. Eyes were cleaned and the epithelium was removed. Eyes were placed on a stand in the middle of a large beaker filled part way with water with a tube bubbling compressed oxygen into the water. The oxygen was turned on at certain times during the experiment to create a humid oxygenated environment for the eye. Eyes were soaked for 20 minutes with 0.1% Riboflavin, dH₂O solution in an incubator set at 37° C. by using a rubber ring to hold the solution on top. Corneas were pan-corneally irradiated with a top hat beam (3% root mean square) for a determined amount of time and irradiance (3 minutes CW at 30 mW/cm² with one drop of solution added every 30 seconds, or 30 minutes CW at 3 mW/cm² with one drop of solution added every minute, or 9 minutes for pulsed light—1.5 seconds on/3 seconds off—using a shutter system (Lambda S.C. Smart Shutter, Sutter Instrument, Novato, Calif.) at 30 mW/cm² with one drop of solution added every minute) with a 365-nm light source (UV LED NCSU033B[T]; Nichia Co., Tokushima, Japan). The irradiance was measured with a power sensor (model PD-300-UV; Ophir, Inc., Jerusalem, Israel) at the corneal surface.

Corneal flaps (approximately 380 μm thick) were excised from the eyes with aid of Intralase femtosecond laser (Abbott Medical Optics, Santa Ana, Calif.). The average thickness of the corneal flaps was calculated as a difference between the measurements before and after the excision from the eyes with an ultrasonic Pachymeter (DGH Technology, Exton, Pa.). The flaps were washed with distilled water 15 times and then dried in a vacuum until the weight change became less than 10% (Rotary vane vacuum pump RV3 A652-01-903, BOC Edwards, West Sussex, UK). Each flap (2 mg) was digested for 2.5 h at 65° C. with 2.5 units/ml of papain (from Papaya latex, Sigma) in 1 ml of papain buffer [1×PBS (pH 7.4), 2 mM L-cysteine and 2 mM EDTA]. Papain digests were centrifuged for 20 seconds at 2200×G (Mini centrifuge 05-090-100, Fisher Scientific), diluted 0.5 times with 1×PBS solution (in other words, 0.5 mL of PBS was added to 1 mL of solution) and fluorescence of the solutions was measured with excitation of λex=360 nm in a QM-40 Spectrofluorometer (Photon Technology Int., London, Ontario, Canada). The fluorescence of the papain buffer was taken into account by measuring fluorescence in the absence of tissue and subtracting this value from the fluorescence of the samples.

### Results

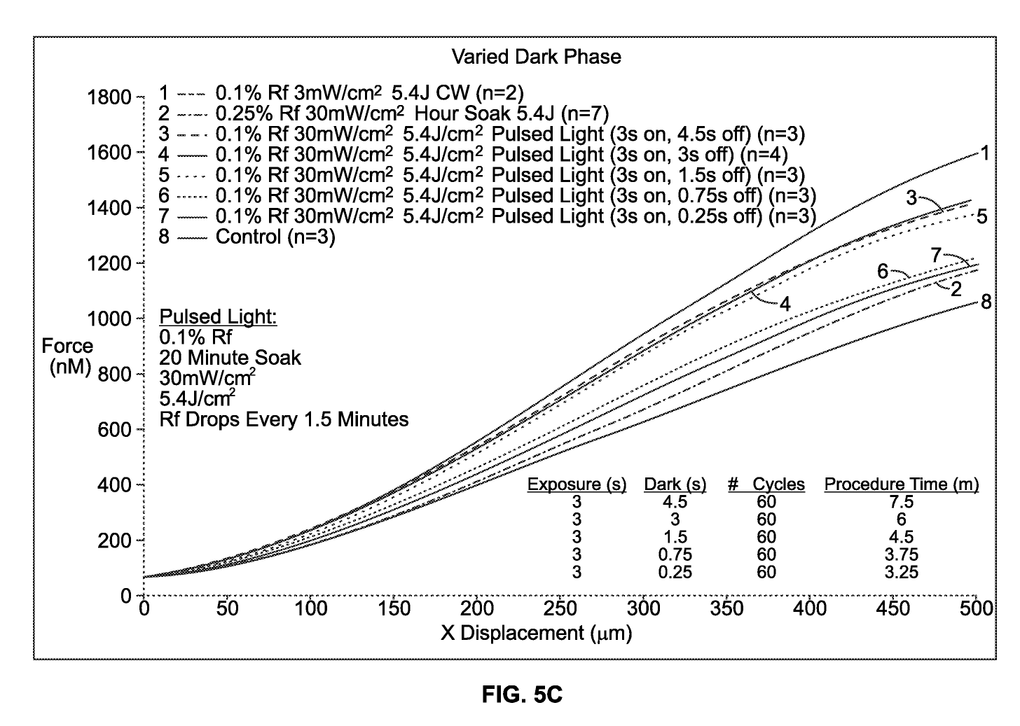

FIG. 7A illustrates a graph of the amount of cross-linking measured by fluorescence of the digested corneal flaps at 450 nm. Eyes were de-epithelized, soaked for 20 minutes with 0.1% Riboflavin, put in a regular air environment or a humid oxygenated environment, and illuminated with 30 mW/cm² or 3 mW/cm² of UV light for 3 minutes CW with one drop of solution every 30 seconds, or 30 minutes CW with one drop of solution every minute, or 9 minutes pulsed (1.5 seconds on/3 seconds off) with one drop of solution every minute. The following treatments shown in TABLE 1 below were applied to each of the samples shown in FIG. 7A:

TABLE 1
Treatment Conditions for the Samples Shown in FIG. 7A

| Sample | Treatment |
|---|---|
| Control | After being soaked, corneal flaps were cut at approximately 380 μm. |
| UV Control | Eyes were illuminated with 30 mW/cm² CW of UV light for 3 minutes. |
| Air, Pulsed Light | Eyes were illuminated with 30 mW/cm² pulsed UV light (1.5 seconds on/ |
| | 3 seconds off) for 9 minutes. |
| O₂, CW | Eyes were illuminated with 30 mW CW/cm² of UV light for 3 minutes |
| | with oxygen always on during UV exposure and soak. |
| 3 mW CW | Eyes were illuminated with 3 mW/cm² CW of UV light for 30 minutes. |
| O₂, Pulsed Light | Eyes were illuminated with 30 mW/cm² of pulsed UV light (1.5 seconds |
| | on/3 seconds off) for 9 minutes with oxygen on during soak and UV |
| | exposure. |

As shown in FIG. 7A, a humid oxygenated environment with pulsed UV light greatly increases the amount of cross-linking taking place in the cornea. Applying a combination of Riboflavin and ultraviolet (UV) light sterilizes a surface of the cornea. The Riboflavin acts as a photosensitizer that increases the absorption of UV light. The resulting absorption of UV light can induce DNA and RNA lesions, and as a result, is effective in killing viruses, bacteria, and other pathogens in the field.

A humid oxygen environment and pulsing UV light increase the amount of cross-linking to a certain degree when done separately, while they increase the amount of cross-linking to a significantly greater extent when done in conjunction. Increased cross-linking involves creation of an increased number of radicals. Radicals help to eliminate harmful bacteria present in the eye. Accordingly, a humid oxygenated environment and pulsing UV light result in more efficient elimination of viruses, bacteria, and other pathogens in the cornea, creating a sterile environment while minimizing any damage or other unwanted effects in the tissue. This is yet another example demonstrating that applying pulsed light illumination as opposed to continuous wave illumination affects the amount of cross-linking in the cornea.

FIG. 7B illustrates force versus displacement curves for samples of porcine cornea illuminated with pulsed light of irradiance with oxygen, as well as curves for samples irradiated with continuous illumination, and a curve for a control sample. The sample 3 irradiated with 30 mW/cm² pulsed light illumination with oxygen and a 3 seconds on/3 seconds off duty cycle had the highest biomechanical stiffness, followed by sample 4 irradiated with 45 mW/cm² pulsed light illumination with oxygen and an identical on/off duty cycle. The samples irradiated with 3 mW/cm² and 30 mW/cm² continuous wave illumination had lower biomechanical stiffness, followed by the control sample. Thus, the addition of oxygen affects the amount of corneal stiffening even at the same on/off duty cycle. This is yet one more parameter that may be altered in optimizing cross-linking.

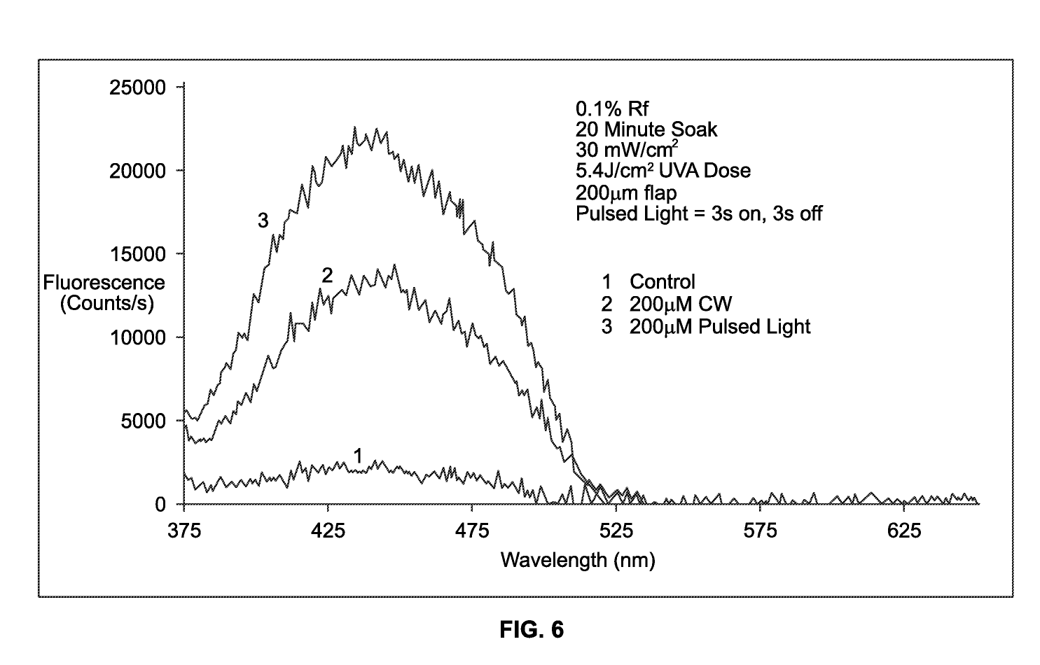

Referring to FIG. 8, a treatment, such as LASIK surgery, is applied in step 210 to generate structural changes in the cornea and produce a desired shape change. In step 220, the corneal tissue is treated with a cross-linking agent 222. The cross-linking agent may be applied directly on the treated tissue and/or in areas around the treated tissue. In some embodiments, the cross-linking agent may be an ophthalmic solution that is broadly delivered by a dropper, syringe, or the like. Alternatively, the cross-linking agent may be selectively applied as an ophthalmic ointment with an appropriate ointment applicator. The cross-linking agent 222 is then activated in step 230 with an initiating element 232. Activation of the cross-linking agent 222, for example, may be triggered thermally by the application of microwaves or light from a corresponding energy or light source. The resulting cross-linking between collagen fibrils provides resistance to changes in corneal structure.

As FIG. 8 shows further, Riboflavin is applied as a cross-linking agent 222 to the corneal tissue in step 220. In addition, light from an UV light source may be applied as an initiating element 232 in step 230 to initiate cross-linking in the corneal areas treated with Riboflavin. Specifically, the UV light initiates cross-linking activity according to the mechanisms described above.

Aspects of the present disclosure relate to monitoring and optimizing the parameters of applying the cross-linking agent to the eye and of activating the cross-linking agent. A large variety of factors affect the rate of the cross-linking reaction and the amount of biomechanical stiffness achieved due to cross-linking. These factors include Riboflavin concentration, conditions on the cornea, temperature, presence of oxidizing agents, the type of illumination applied to activate the Riboflavin, the irradiance, the dose, the on/off duty cycle of the applied illumination, as well as other factors. A number of these factors are interrelated, in other words, changing one factor may have an unexpected effect on another factor.

Aspects of the present disclosure relate to determining the effect of each of these parameters on the rate and the amount of cross-linking, as well as the interrelations of these parameters among each to optimize the conditions to achieve the desired amount, rate, and location of corneal stiffening. Aspects of the present disclosure relate to monitoring the corneal response to a change in one or a plurality of parameters and adjusting the one or the plurality of parameters based on the received feedback.

Although the embodiments described above may employ stepwise on/off pulsed light functions, it is understood that other functions for applying light to the cornea may be employed to achieve similar effects. For example, light may be applied to the cornea according to a sinusoidal function, sawtooth function, or other complex functions or curves, or any combination of functions or curves. Indeed, it is understood that the function may be “substantially” stepwise where there may be more gradual transitions between on/off values. In addition, it is understood that irradiance does not have to decrease down to a value of zero during the off cycle, and may be above zero during the off cycle. Effects of the present disclosure may be achieved by applying light to the cornea according to a curve varying irradiance between two or more values.

Current monochromatic CW cross-linking provides for limited user control of either (1) a target depth within the eye; (2) the density of cross-links; or (3) the particular method (energy or electron transfer) of cross-linking. Further to patterned (2D/DMD) actinic excitation with real time fluorescence dosimetry feedback, there is a need to provide for accelerated cross-linking of tissue at (1) a user selectable depth and (2) to generate a user-targeted cross-link density profile therein with a controlled amount of apostosis introduction. Such control may allow for customization of a treatment based on precise patient anatomy matched corneal refractive reshaping in a safe and accelerated manner.

Cross-linking is known to require suitable concentrations of agents including actinic radiance, photosensitizer, and dissolved oxygen for radicals generation and maintenance near the targeted tissue/protein substrate for the duration of the treatment. The concentrations of these agents change in tissue with depth. One of the aspects of the present invention relates to formulating extensive relationships where the rates/concentrations of photo-bleaching, oxygen consumption/radicals generation, and photosensitizer/oxygen re-diffusion are used to construct multi-wavelength pulsing regimes (i.e., irradiance/duty cycle/synchronizing wavelength exposure) based on real time feedback of oxygen and photosensitizer consumption and concentrations as a function of depth. For example, an optimized way of delivery of the agents (e.g., actinic radiance, photosensitizer, dissolved oxygen) may include maximal oxygen pre-loading of the photosensitizers, multiple wavelength matching a mix of photosensitizers or a single photosensitizer for calibrated radical generation and potentially using elasticity mapping feedback for 3D pattern optimization. The localized cross-linking exposure duration required is approximated by the experimentally determined rate equations and can be factored into the delivery (pulsing) protocol, so that the system can target for depth and degree of cross-linking.

According to one aspect of the present invention, the delivery system of FIG. 1 is modified such that the light source 110 includes multi-wavelength LED printed circuit boards (PCBs) and achromatic optics.

### Example 6

A patient presents with a 300-350 μm thick cornea and is desirous of a 1.25 D spherical flattening having 0.5 D cylinder (manifest refraction spherical equivalents (MRSE)). Bio-mechanical pre-screening reveals no concerns for ectasia with elasticity map in the ranges of 150 kPa to 250 kPa; topography shows a 0.75 D cylinder ATR as well.

A modeling analysis based nomogram generator (which combines elasto-fine element analysis and accelerated sub-surface cross-link density modulation) prescribes, for example, a UVA Riboflavin treatment in the central (5 mm) zone for a 200 μm treatment depth and a 1 mm transition zone in the periphery at 250 μm depth where the cornea is thicker. An average of 20 μm shrinkage is targeted. A pulsed spot illumination with fluorescence dosimetry feedback is implemented so that the dosimetry depth profile confirms adequate Riboflavin concentration at 200-250 μm and is sequenced by a bowtie pattern for the 0.75 D cylinder correction. The shape/axis of the bowtie is entirely derived from the topography and is registered by an iris tracker during treatment. Delivery may be topographically guided or topographically optimized.

As discussed above, one of the aspects of the present invention relates to creating more efficient and controlled cross-link densities at depth. Several sequences that perform feedback-controlled micro-volume cross-link sub-steps when attempting a bulk exposure were evaluated.

An example regime includes:

1 to N pulses (pulse train) at a wavelength λ, followed by 1 to N pulses at a wavelength B, followed by 1 to N pulses at a wavelength C. Alternatively, each sub-step of the sequence may include a different number of pulses.

The duration of each exposure may vary between the pulse trains. The duration of a dark period following each exposure may vary as well. Alternatively, there may be no dark periods or pauses in between different pulse trains.

The wavelength may vary between the pulse trains. The wavelength may increase for each pulse train, decrease for each pulse train, or the wavelength may be alternated between the different pulse trains. Alternatively, the wavelength may be selected based on a predetermined pattern.

The irradiance may vary between the pulse trains. The irradiance may increase for each pulse train, decrease for each pulse train, or the irradiance may be alternated between the different pulse trains. Alternatively, the irradiance may be selected based on a predetermined pattern.

According to one aspect, a sequence of pulse trains is applied, followed by a pause of a predetermined duration, and followed by an application of another sequence of pulse trains.

According to a further aspect, a sequence includes a brief sequence of high irradiance pulses followed by lower irradiance pulses repeating while under maximally hyperoxic conditions.

The tissue may be inducted by any sequence of oxygen in normoxic, hyperoxic, or hypoxic conditions, followed by addition of nitrogen prior to or during any of the pulsing train sequences discussed above.

Photosensitizers including Riboflavin and Indocyanine green (ICG) may be used in combination with any of the sequences discussed above.

The sequence is dynamic and may be adjusted depending on the desired effect and individual patient characteristics. Near infrared light (NIR), for example 904 nm light, generates reactive oxygen species with only endogenous tissue molecules and can be used as an in vivo reactive oxygen species generator simultaneously with the other illumination (UVA, etc.).

Formulations may include agents (chaperones) configured to improve wound healing and to protect the eye from unwanted side effects such as apoptosis or haze.

Pulsing parameters may be selected based on target micro-volume fluorescence to increase safety and efficiency. Zonal bleaching based bulk oxygenation can also be used for increased safety and efficiency.

One or more chemical accelerators and/or chemical quenchers (such as ascorbic acid) of various concentrations may be added before and/or during the pulse sequence to control the depth of cross-linking. Various quenchers and quenching methods are discussed in U.S. patent application Ser. No. 13/475,175, filed May 18, 2012 the disclosure of which is incorporated entirely herein by reference.

Rinsing with hyper, hypo or isotonic non-photo reactive solutions may also be used to vary the concentration profile of the photosensitizer within the tissue. In particular, rinsing may protect against damage to the superficial corneal nerve plexus which is known to be destroyed following conventional cross-linking.

Any combination of the permutations discussed above may be applied to achieve a desired effect of creating a more efficient and/or specific cross-linking profile through the tissue. Moreover, different parameters may be varied to achieve a desired depth and density of cross-linking. Furthermore, different parameters may be varied to achieve a particular method of cross-linking. The different parameters that may be varied include wavelength, irradiance, duration, on/off duty cycle, oxygenation conditions in the tissue, photosensitizer selection, presence of additional agents and solutions.

Generally, eye treatments, such as LASIK surgery, involve procedures to the anterior corneal tissue. While the procedures achieve a direct change in the shape of the anterior corneal tissue, the posterior corneal tissue generally does not change shape in a corresponding fashion. Accordingly, after such procedures, the posterior corneal tissue may exert a force on the anterior corneal tissue that counters or inhibits the desired changes to the corneal tissue affected by the procedures. The forces applied by the posterior corneal tissue on the anterior corneal tissue may prevent the procedure from achieving the desired structural change. As a result, for example, more severe ablation of corneal tissue, greater amounts of thermal energy, and/or greater amounts of cross-linking agents may be required to account for the force applied by the posterior corneal tissue on the anterior corneal tissue and achieve a desired change to the corneal tissue.

Embodiments also relate to systems and processes for conducting an eye treatment that address such problems. In particular, embodiments involve a procedure to cut one or more dissection planes or regions in the cornea to at least partially disassociate or separate the anterior corneal tissue from the posterior corneal tissue to provide one or more areas of stress relief. By providing one or more areas of stress relief, embodiments reduce the extent of eye treatment required to achieve a desired change in corneal tissue and improve the stability of changes to the corneal tissue as part of eye treatment.

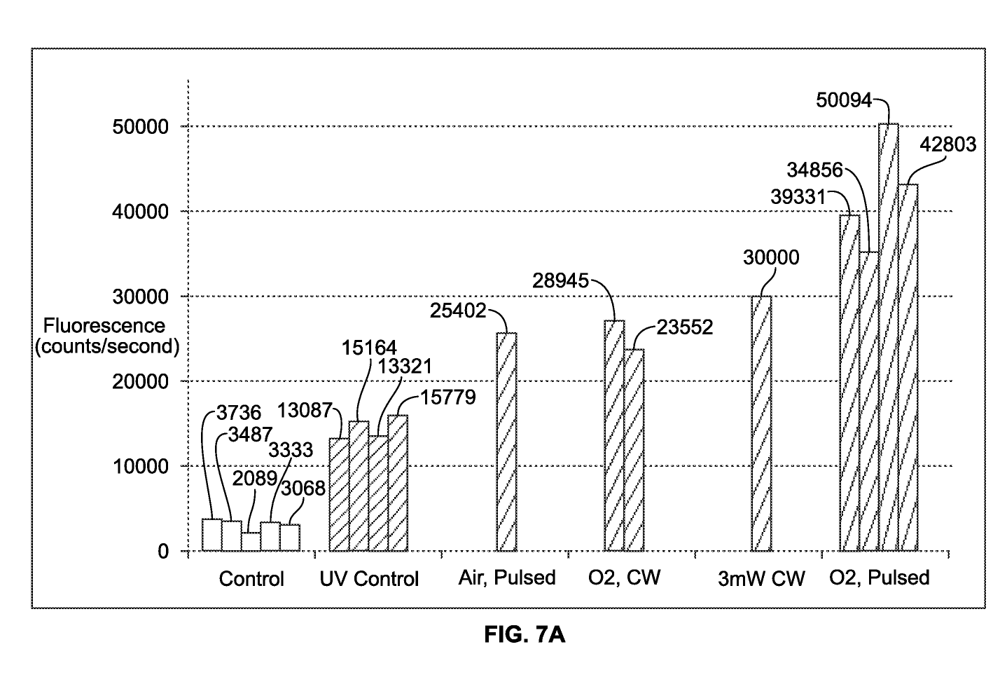

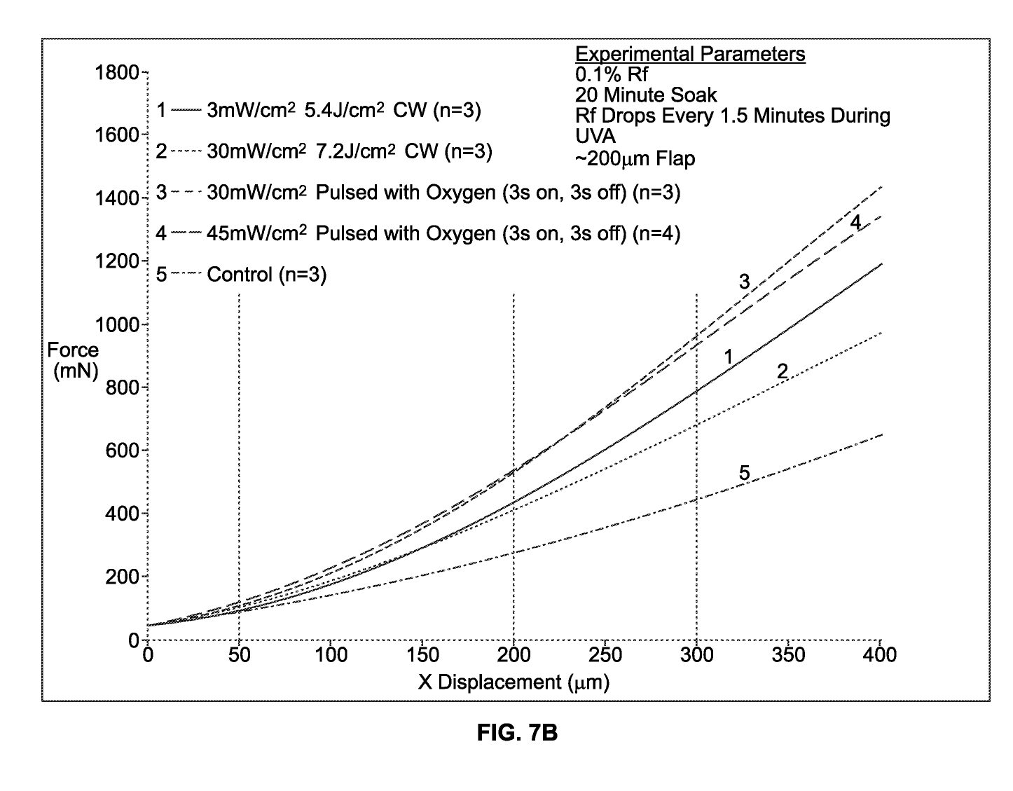

FIG. 9 illustrates a cornea 2 of an eye 1, including an anterior corneal tissue 2C and a posterior corneal tissue 2D. FIG. 10 illustrates an example process 500 for performing a treatment on an eye. In step 510, the anatomy of a patient's eye 1 is determined using a measurement device. The determination of the eye 1 anatomy may include, for example, a determination of the curvature and the thickness of the anterior corneal tissue 2C and the posterior corneal tissue 2D. Non-limiting examples of measurement devices that are suitable to assist in determining the anatomy of the eye 1 include a tonometer, an ultrasound pachymeter, an optical pachymeter, and/or an imaging device. Aspects of systems and approaches for making such measurements are described in U.S. application Ser. No. 13/051,699, filed Mar. 18, 2011, and U.S. application Ser. No. 13/438,705, filed Apr. 12, 2012, referenced above.

At step 520, one or more locations, sizes, and depths are determined for one or more incisions to be formed in the posterior corneal tissue 2C. The locations, sizes, and depths of the one or more incisions to the posterior corneal tissue 2D depend on the anatomical structure of the patient's eye (e.g., cornea 2), the particular optical condition that is to be corrected (e.g., myopia, keratoconus, or hyperopia), and/or the type of eye treatment to be applied (e.g., themokeratoplasty or LASIK) to reshape the cornea 2. In particular, the location, size, and depth of the one or more incisions are determined so as to at least partially disassociate or separate the anterior corneal tissue 2C from the posterior corneal tissue 2D without weakening the structural integrity of the eye 1. Accordingly, the one or more incisions may take the form of one or more dissection planes or regions. The one or more dissection planes or regions can be optimized for particular applications by, for example, localizing the one or more incisions to a specific region or providing the one or more incisions in a particular pattern depending on the anatomical structure of the patient's eye, the optical condition corrected, and/or the method of eye treatment employed.

To avoid a weakening of the structural integrity of the eye 1, the location, size, and depth of the one or more incisions are generally determined so that the incisions do not penetrate through the full thickness of the cornea 2 (i.e., from the posterior corneal tissue 2D through the anterior corneal tissue 2C). In some embodiments, it is contemplated that the one or more incisions may be determined to have a location, size, and depth such that the one or more incisions formed in the posterior corneal tissue 2D do not penetrate into any portion of the anterior corneal tissue 2C. According to some embodiments, the one or more locations, sizes, and depths for the one or more incisions may be determined and/or optimized by a controller (e.g., a computer processing system that reads instructions on computer-readable storage media).

At step 530, the one or more incisions are formed in the posterior corneal tissue 2 by an incision device according to the one or more locations, sizes, and depths determined at step 520. As a non-limiting example, the incision device can be a femtosecond pulsed laser that is configured or controlled (e.g., by one or more controllers) to form the desired one or more incisions. The one or more incisions at least partially disassociate or separate the posterior corneal tissue 2D from the anterior corneal tissue 2C so as to provide for one or more areas of stress relief.

An eye treatment (e.g., LASIK surgery or cross-linking treatment) is applied at step 540 to generate structural changes in the anterior corneal tissue 2C and produce a desired shape change. The system for applying the eye treatment may include any device that is suitable for applying, for example, LASIK surgery. One non-limiting example of a device for applying LASIK is an excimer laser.

Advantageously, the eye treatment applied to the eye 1 may take into account the reduced forces that the posterior corneal tissue 2D exerts on the anterior corneal tissue 2C due to the one or more incisions. As a result, the extent of eye treatment required to achieve a desired change in corneal tissue may be reduced. For example, in a LASIK eye treatment procedure, a more moderate ablation of anterior corneal tissue 2C may be required to achieve a desired change in the corneal shape. Likewise, in a cross-linking treatment, a reduced amount of cross-linking agent or lower dose of UV light may be required to achieve a desired reshaping of the corneal shape. Accordingly, the precise amount of treatment to be applied to the eye (e.g., laser ablation, magnitude of electrical energy, size of electrical energy pattern, number of electrical pulses, amount of cross-linking agent, and/or dose of UV light) can be determined and controlled by one or more controllers that take into account the anatomy of the patient's eye and the one or more incisions to the posterior corneal tissue 2D.

Additionally, after the application of the eye treatment at step 540, the resulting shape of the anterior corneal tissue 2C may exhibit greater stability as the one or more incisions provide area(s) of stress relief against the forces applied by the posterior corneal tissue 2D to the anterior corneal tissue 2C. Optionally, at step 550, a cross-linking agent can be further applied to the cornea 2 to stabilize the corneal tissue 2 and improve its biomechanical strength, e.g., in combination with LASIK surgery, as described above.

The embodiment described with reference to FIG. 5 provides an example in which incisions are employed to promote desired shape change in corneal structure. Indeed, it is contemplated that such incisions are not limited to posterior corneal tissue. In general, a cutting instrument, such as a femtosecond laser, may be employed to make incisions in any portion of the cornea to create slip planes that allow aspects of the corneal structure to move more easily relative to each other and to allow desired reshaping to take place when combined with other eye treatments, such as LASIK surgery or cross-linking treatment. Indeed, it is contemplated that some particular shape changes would not be otherwise possible without the creation of one or more slip planes. The location, size, depth of the slip planes depends on the desired shape change.

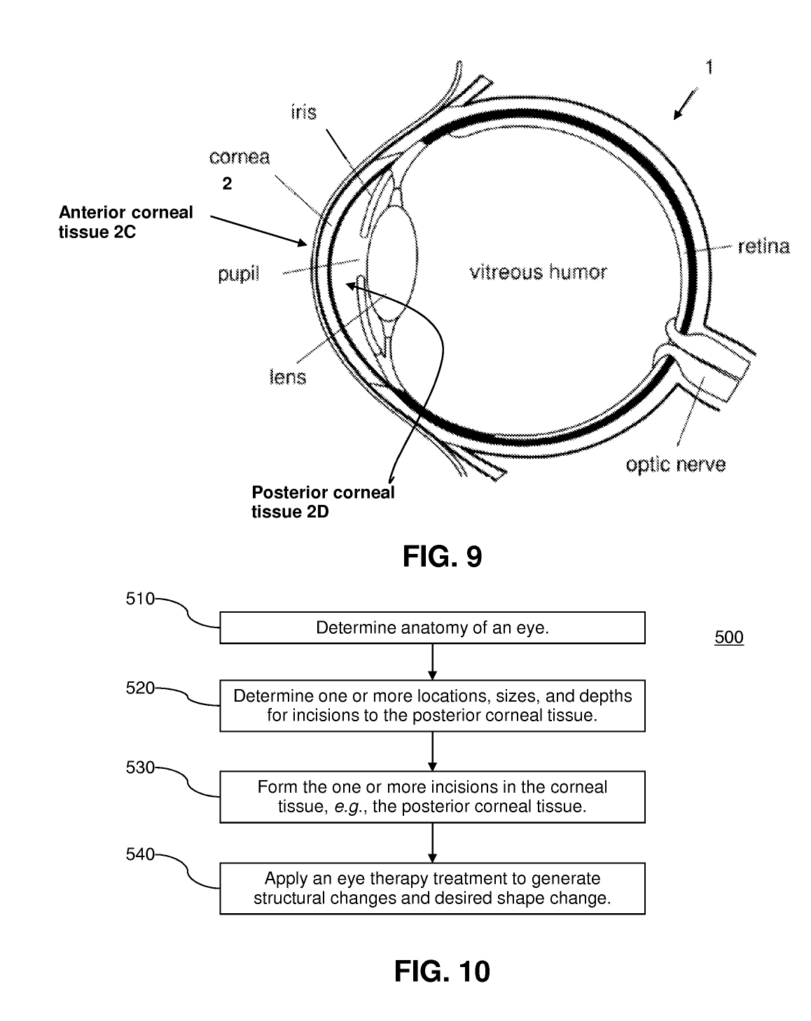

FIG. 11 illustrates an example integrated system 600, in which the components can be employed to manipulate varying aspects of the corneal structure in order to achieve customized shape change. In particular, a cutting instrument 610, e.g., femtosecond laser, is combined with at least one eye treatment system: a LASIK surgery system 620 and/or the cross-linking system 100 as described with reference to FIG. 1. The components of the system 600 can be controlled by one or more controllers 640, which make measurements, provide monitoring, and/or drive the components, e.g., based on feedback from the monitoring.

Thus, in operation, the cutting instrument is employed to create incisions in selected areas of the cornea. One of the eye therapy systems applies reshaping forces to the cornea. For example, the LASIK surgery system 620 ablates the corneal tissue with an excimer laser to apply the reshaping forces after a microkeratome creates a corneal flap or the cross-linking treatment system 100 applies a cross-linking agent, e.g., Riboflavin, and photoactivating light, e.g., UV light, to initiate cross-linking activity in selected areas of the cornea and apply the reshaping forces. The controller(s) 640 can determine the selected areas of the cornea for the incisions and the reshaping forces from the eye therapy system, such that the reshaping forces and the incisions combine to achieve a predetermined corrective reshaping of the cornea.

As described above, the cross-linking system 100, e.g., through the controllers 640, may control one or more parameters to achieve the desired amount, rate, and location (on the cornea 2) of corneal stiffening. For example, the controller(s) 640 may be employed to control with precision the delivery of photoactivating light to the cornea 2 by operating the corresponding optical elements according to any combination of: wavelength, bandwidth, intensity, power, location, depth of penetration, and duration (the duration of the exposure cycle, the dark cycle, and the ratio of the exposure cycle to the dark cycle duration) of treatment.

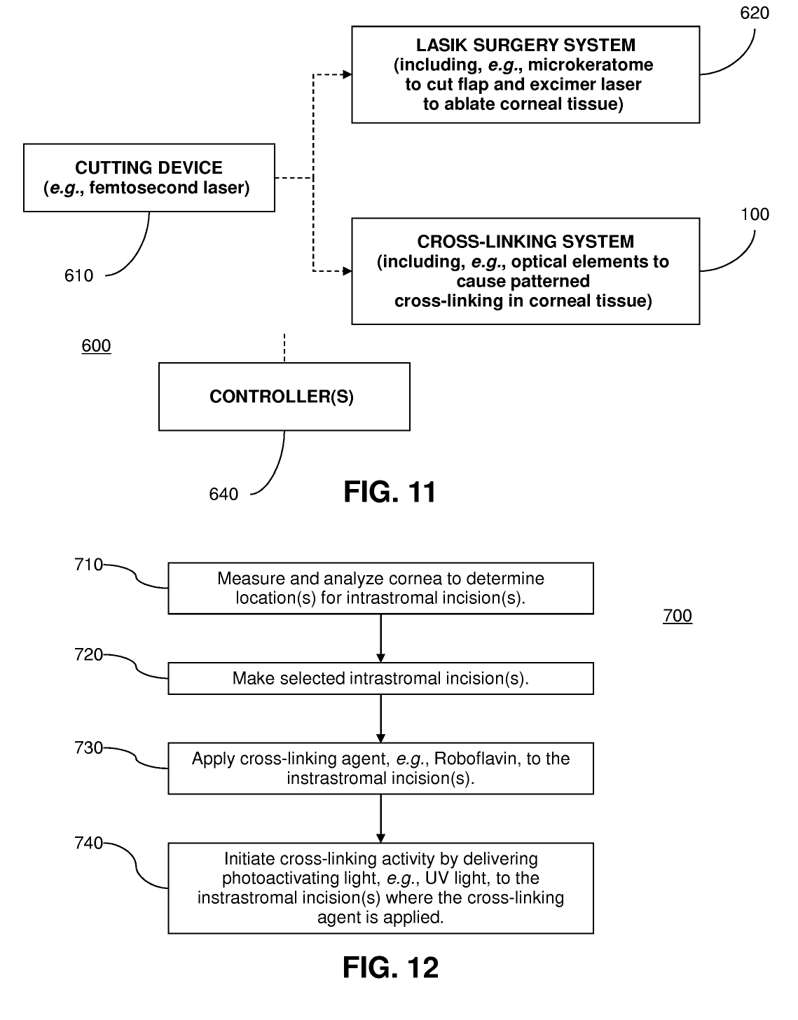

Aspects of the cross-linking treatments described herein may be applied in combination with any eye therapy that may require additional stabilization of the corneal tissue. In particular, the eye therapy may include selective incision of one or more sections of the cornea. Thus, in further embodiments, cross-linking treatments are combined with astigmatic keratotomy eye surgery (AK). AK is a surgical procedure for correcting astigmatism, where incisions are made in the steepest part of the abnormally shaped cornea to relax the cornea into a rounded shape. AK is often employed in combination with cataract surgery. The resulting shape change from AK can be stabilized with cross-linking treatment. A form of AK is called intrastromal astigmatic keratotomy (ISAK). In the example process 700 shown in FIG. 12, ISAK is performed in steps 710 and 720. In particular, in step 710, a cornea with astigmatism is measured and analyzed to determine the location(s) for intrastromal incision(s) that will relax the cornea into the desired shape. In step 720, a femtosecond laser is used to make incisions in the stroma of the cornea without breaking Bowman's or Descemet's membranes. The length, height, and shape (e.g., arc radius) of the incisions, for example, may vary to achieve the desired corneal shape. In step 730, Riboflavin of a known concentration and quantity is then injected into the intrastromal incisions with a tiny needle and allowed to soak for a prescribed amount of time. In step 740, cross-linking activity is then initiated where photoactivating light may be titrated to get variable amounts of energy delivered to control the amount of astigmatic correction desired. The concentration and soak time for the application of Riboflavin and the irradiance, dose, and patterning for the delivery of the photoactivating light, for example, may be varied to achieve the required amount of cross-linking.

Similarly, cross-linking treatments may be combined with radial keratotomy (RK), which is a surgical procedure for correcting myopia, where radial incisions are made to the cornea to make corrective shape changes. In addition to stabilizing the corrective shape changes, the effect of applying the cross-linking agent may also allow smaller incisions to be used during AK or RK.

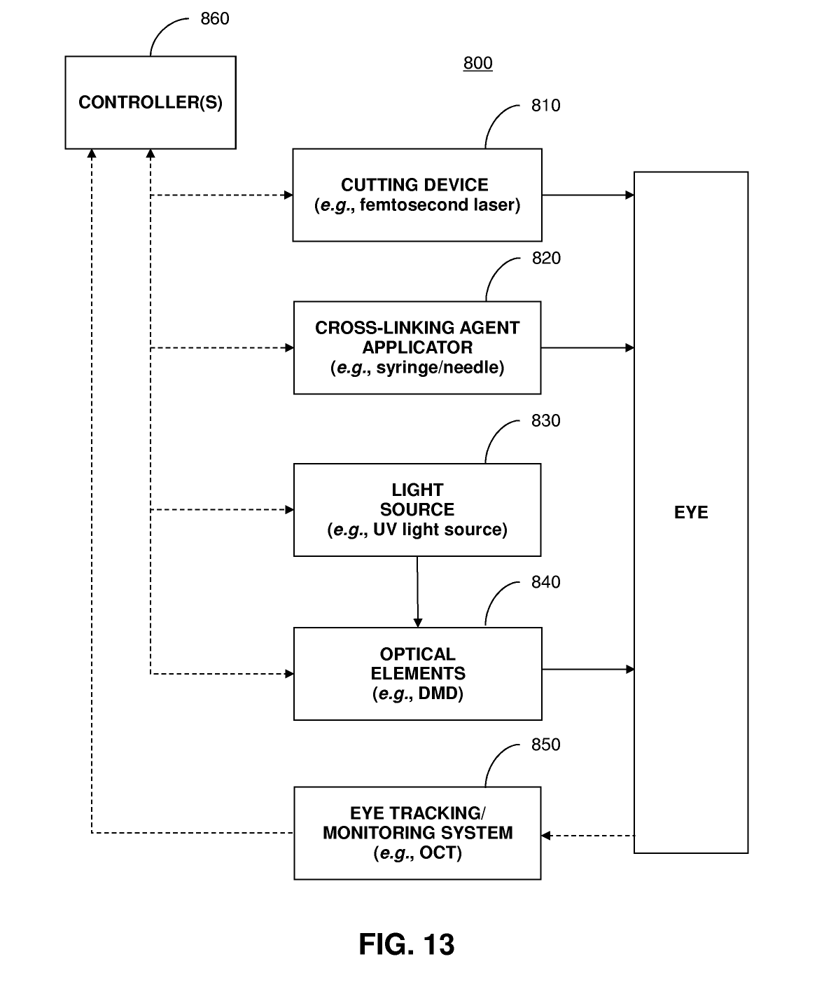

FIG. 13 illustrates an example system 800 that can be employed to apply cross-linking treatments with AK or RK, according to aspects of the present invention. A cutting device 810, such as a femtosecond laser, is employed to create the desired incisions to the corneal tissue to make corrective shape changes according to AK or RK. A cross-linking agent applicator 820, such as a syringe or needle, is employed to apply cross-linking agent (e.g., Riboflavin) to the area of the incisions. Optical elements 840, such as a digital micro-mirror device (DMD) and/or other devices described herein, are employed to deliver photoactivating light from a light source 830, such as a UV light source, to the area of the incisions. The optical elements 840 allow the photoactivating light to be precisely and accurately delivered to the areas treated with the cross-linking agent and to initiate the desired amount of cross-linking activity as described above. One or more controllers 860 may be employed to control the operation of aspects of the system 800. For example, the controller(s) 860 may control the optical elements 840 to deliver photoactivating light according to any combination of: wavelength, bandwidth, intensity, power, location, depth of penetration, and duration (the duration of the exposure cycle, the dark cycle, and the ratio of the exposure cycle to the dark cycle duration) of treatment. An eye-tracking and/or monitoring system 850 may be employed to provide feedback to the controller(s) 860 to control dynamically the progress of the incisions, the application of the cross-linking agent, and/or the application of the photoctivating light. For example, the pattern of the photoactivating light may be controlled through eye tracking and real-time monitoring of the procedure through fluorescence dosimetry, optical coherence tomography (OCT), interferometry, abberometry, etc.

In the embodiments described herein, systems may include one or more controllers (e.g., a computer processing system that reads instructions on computer-readable storage media) to process the information determined for the anatomy of the eye, determine the locations, sizes, and depths for incisions to the corneal tissue, control the incision device in forming the incisions, and/or control the eye treatment systems in applying the eye treatment to the eye. Generally, the one or more controllers may be implemented as a combination of hardware and software elements. The hardware aspects may include combinations of operatively coupled hardware components including microprocessors, logical circuitry, communication/networking ports, digital filters, memory, or logical circuitry. The one or more controllers may be adapted to perform operations specified by a computer-executable code, which may be stored on a computer readable medium.

As described above, the one or more controllers may be a programmable processing device, such as an external conventional computer or an on-board field programmable gate array (FPGA) or digital signal processor (DSP) that executes software, or stored instructions. In general, physical processors and/or machines employed by embodiments for any processing or evaluation may include one or more networked or non-networked general purpose computer systems, microprocessors, field programmable gate arrays (FPGA's), digital signal processors (DSP's), micro-controllers, and the like, programmed according to the teachings of the exemplary embodiments, as is appreciated by those skilled in the computer and software arts. The physical processors and/or machines may be externally networked with the image capture device(s), or may be integrated to reside within the image capture device. Appropriate software can be readily prepared by programmers of ordinary skill based on the teachings of the exemplary embodiments, as is appreciated by those skilled in the software art. In addition, the devices and subsystems of the exemplary embodiments can be implemented by the preparation of application-specific integrated circuits or by interconnecting an appropriate network of conventional component circuits, as is appreciated by those skilled in the electrical art(s). Thus, the exemplary embodiments are not limited to any specific combination of hardware circuitry and/or software.

Stored on any one or on a combination of computer readable media, the exemplary embodiments may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, and the like. Such computer readable media further can include the computer program product of an embodiment for performing all or a portion (if processing is distributed) of the processing performed in implementations. Computer code devices of exemplary embodiments can include any suitable interpretable or executable code mechanism, including but not limited to scripts, interpretable programs, dynamic link libraries (DLLs), Java classes and applets, complete executable programs, and the like. Moreover, parts of the processing of exemplary embodiments can be distributed for better performance, reliability, cost, and the like.

Common forms of computer-readable media may include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

Although embodiments have been described in connection with LASIK surgery, or the like, it is understood that the systems and methods described may be applied with other eye treatments.

Although the embodiments described above may employ Riboflavin as a cross-linking agent, it is understood that other substances may be employed as a cross-linking agent. Thus, for example Rose Bengal (4,5,6,7-tetrachloro-2′,4′,5′,7′-tetraiodofluorescein) may be employed as cross-linking agent. Rose Bengal has been approved for application to the eye as a stain to identify damage to conjunctival and corneal cells. However, Rose Bengal can also initiate cross-linking activity within corneal collagen to stabilize the corneal tissue and improve its biomechanical strength. Like Riboflavin, photoactivating light may be applied to initiate cross-linking activity by causing the Rose Bengal to form radicals and to convert O₂ in the corneal tissue into singlet oxygen. The photoactivating light may include, for example, UV light or green light.

While the present disclosure has been described in connection with a number of exemplary embodiments, and implementations, the present disclosure is not so limited, but rather covers various modifications, and equivalent arrangements. In addition, although aspects of the present invention may be described in separate embodiments, it is contemplated that the features from more than one embodiment described herein may be combined into a single embodiment.

What is claimed is:

1. A system for treating an eye, comprising:
a cutting instrument configured to make incisions in selected areas of the cornea;
a cross-linking treatment system, the cross-linking treatment system including an applicator that applies a cross-linking agent to the selected areas of the cornea, a light source that provides photoactivating light for the cross-linking agent, and one or more optical elements that deliver the photoactivating light to the selected areas of the cornea, the photoactivating light acting on the cross-linking agent to initiate cross-linking activity in the selected areas of the cornea; and
one or more controllers that determine the selected areas of the cornea for the incisions according to astigmatic keratotomy or radial keratotomy.

2. The system of claim 1, wherein the cutting instrument includes a femtosecond laser.

3. The system of claim 1, wherein the applicator includes a needle configured to apply the cross-linking agent to the incisions.

4. The system of claim 1, wherein the cross-linking agent includes Riboflavin and the photoactivating light is ultraviolet light.

5. The system of claim 1, further comprising a monitoring system that provides feedback to the one or more controllers on the incisions made by the cutting instrument and the

6. A method for treating an eye, comprising:
selecting locations for making incisions in areas of the cornea according to astigmatic keratotomy or radial keratotomy;
making, with a cutting instrument, incisions in the selected areas of the cornea;
applying, with an applicator, a cross-linking agent to the selected areas of the cornea; and
delivering, with one or more optical elements, photoactivating light from a light source to the selected areas of the cornea to initiate cross-linking activity in the selected areas of the cornea.

7. The method of claim 6, wherein the cutting instrument includes a femtosecond laser.

8. The system of claim 6, wherein the applicator includes a needle configured to apply the cross-linking agent to the incisions.

9. The method of claim 6 wherein the cross-linking agent includes Riboflavin and the photoactivating light is ultraviolet light.
